SEQUENCE LISTING (36 sequences)

SEQ ID NO: 1 - DNA, 910 bp, Homo sapiens
acaggaggctgcttagccacatgggaggtgctcagtaaaggagagcaattcttacaggtgtctgcctcctgacccttccatccctcaggtgtcctgttgccccctcctcccactgacaccctccggaggcccccatgttgacagaccctccttctcctaccttgtttcccagcctgactctccttccgttctgggtccccctcctctggtcggctcccctgtgtctcatcccccggattaagccttctccgcctggtcctctttctctggtgacccacaccgcccgcaaagccacagcgcatctggatcacccgctttggtggcgcttggccgccaggaggcagcaccctgtttgcggggcggagccggggagcccgccccctttcccccagggctgaagggacccccctcggagcccgcccacgcgagatgaggacggtggcccagcccccccatgccctccccctgggggccgcccccgctcccgccccgtgcgcttcctgggtggggccgggggcggcttcaaaaccccctgccgacccagccggtccccgccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgcccc

SEQ ID NO: 2 - DNA, 28 bp, Homo sapiens
aaggagagcaattcttacaggtgtctgc

SEQ ID NO: 3 - DNA, 7 bp, Homo sapiens
ttcttac

SEQ ID NO: 4 - DNA, 29 bp, Homo sapiens
ttcccagcctgactctccttccgttctgg

SEQ ID NO: 5 - DNA, 7 bp, Homo sapiens
actctcc

SEQ ID NO: 6 - DNA, 30 bp, Homo sapiens
gtcggctcccctgtgtctcatcccccggat

SEQ ID NO: 7 - DNA, 8 bp, Homo sapiens
tgtgtctc

SEQ ID NO: 8 - DNA, 30 bp, rat
aaggaaacttgagtcaggtgggcgagagca

SEQ ID NO: 9 - DNA, 8 bp, rat
agtcaggt

SEQ ID NO: 10 - DNA, 700 bp, Homo sapiens
tttactgccctgaagattcagcctgcggaagacagggggttgccccagtggaattccccagccttgcctagcagagcccattccttccgcccccagatgaagcagggagaggaagctgagtcaaagaaggctgtcagggagggaaaaagaggacagagcctggagtgtggggaggggtttggggaggatatctgacctgggagggggtgttgcaaaaggccaaggatgggccagggggatcattagtttcagaaagaagtctcagggagtcttccatcactttcccttggctgaccactggaggctttcagaccaagggatgggggatccctccagcttcatccccctccctccctttcatacagttcccacaagctctgcagtttgcaaaaccctacccctcccctgagggcctgcggtttcctgcgggtctggggtcttgcctgacttggcagtggagactgcgggcagtggagagaggaggaggtggtgtaagccctttctcatgctggtgctgccacacacacacacacacacacacacacacacacacacacacacacaccctgacccctgagtcagcacttgcctgtcaaggaggggtggggtcacaggagcgcctccttaaagcccccacaacagcagctgcagtcagacacctctgccctcaccatgagcctctggcagcccctggtcctgg

SEQ ID NO: 11 - DNA, 30 bp, Homo sapiens
cctgacccctgagtcagcacttgcctgtca

SEQ ID NO: 12 - DNA, 8 bp, Homo sapiens
agtcagca

SEQ ID NO: 13 - DNA, 29 bp, Homo sapiens
ggttgccccagtggaattccccagccttg

SEQ ID NO: 14 - DNA, 7 bp, Homo sapiens
tggaatt

SEQ ID NO: 15 - DNA, 30 bp, rat
gagtcagcgtaagcctggagggaggggcgg

SEQ ID NO: 16 - DNA, 8 bp, rat
agcctgga

SEQ ID NO: 17 - DNA, 28 bp, rat
gggttgccccgtggaattcccccaaatc

SEQ ID NO: 18 - DNA, 6 bp, rat
tggaat

SEQ ID NO: 19 - DNA, 20 bp, artificial sequence
  Synthetic forward primer
ccaaggagacggaatacagg

SEQ ID NO: 20 - DNA, 19 bp, artificial sequence
  Synthetic reverse primer
agctgtgcaggtgttgagc

SEQ ID NO: 21 - DNA, 20 bp, artificial sequence
  Synthetic forward primer
ttcgacgctgacaagaagtg

SEQ ID NO: 22 - DNA, 20 bp, artificial sequence
  Synthetic reverse primer
aggggagtcctcgtggtagt

SEQ ID NO: 23 - DNA, 20 bp, artificial sequence
  Synthetic forward primer
acatcaaatggggtgatgct

SEQ ID NO: 24 - DNA, 20 bp, artificial sequence
  Synthetic reverse primer
gtggttcacacccatcacaa

SEQ ID NO: 25 - DNA, 30 bp, rat
ggaagaaaggaaacttgagtcaggtgggcg

SEQ ID NO: 26 - DNA, 8 bp, rat
aacttgag

SEQ ID NO: 27 - DNA, 50 bp, Rat
gcactggggaagaaaggaaacttgagtcaggtgggcgagagcagagcagg

SEQ ID NO: 28 - DNA, 50 bp, Rat
cctgctctgctctcgcccacctgactcaagtttcctttcttccccagtgc

SEQ ID NO: 29 - DNA, 50 bp, Rat
cacaccctgagtcagcgtaagcctggagggaggggcggggacactgattc

SEQ ID NO: 30 - DNA, 50 bp, Rat
gaatcagtgtccccgcccctccctccaggcttacgctgactcagggtgtg

SEQ ID NO: 31 - DNA, 50 bp, Rat
ctggggttagggggttgccccgtggaattcccccaaatcctgcttcaaag

SEQ ID NO: 32 - DNA, 50 bp, Rat
ctttgaagcaggatttgggggaattccacggggcaaccccctaaccccag

SEQ ID NO: 33 - DNA, 28 bp, Homo sapiens
accctgacccctgagtcagcacttgcct

SEQ ID NO: 34 - DNA, 28 bp, Homo sapiens
aggcaagtgctgactcaggggtcagggt

SEQ ID NO: 35 - DNA, 26 bp, Homo sapiens
tgccccagtggaattccccagccttg

SEQ ID NO: 36 - DNA, 26 bp, Homo sapiens
caaggctggggaattccactggggca

(19) United States
(12) Patent Application Publication
(10) Pub. No.: US 2012/0071628 A1
(43) Pub. Date: Mar. 22, 2012

(54) TOPICAL THERAPEUTIC AGENT FOR OPHTHALMIC DISEASES COMPRISING COMPOUND CAPABLE OF BINDING SPECIFICALLY TO DNA SEQUENCE

(75) Inventors: Hiroki Nagase, Tokyo (JP); Min Chen, Tokyo (JP); Tohru Sakimoto, Tokyo (JP); Takayoshi Watanabe, Tokyo (JP); Noboru Fukuda, Tokyo (JP)

(73) Assignee: NIHON UNIVERSITY, Tokyo (JP)

(21) Appl. No.: 13/256,012

(22) PCT Filed: Sep. 15, 2009
(86) PCT No.: PCT/JP2009/066108

§ 371 Date: Nov. 28, 2011

(30) Foreign Application Priority Data
Mar. 13, 2009 (JP) 2009-062116

(51) Int. Cl. C07K 7/08; C07K 2/00

(52) U.S. Cl. 530/326; 530/300

(57) ABSTRACT

Disclosed is a topical therapeutic agent for ophthalmic diseases, which comprises a compound capable of binding specifically to a DNA sequence. More preferably disclosed is a topical therapeutic agent for ophthalmic diseases, which comprise a pyrrole-imidazole polyamide having a specific structure. The topical therapeutic agent for ophthalmic diseases comprises a pyrrole-imidazole polyamide which can inhibit transforming growth factor-β gene and matrix metalloproteinase 9 gene.

### TECHNICAL FIELD

The present invention relates to a topical therapeutic agent for an ophthalmic disease containing a compound capable of binding specifically to a DNA sequence. More specifically, the present invention relates to a topical therapeutic agent for an ophthalmic disease containing a pyrrole-imidazole polyamide (hereinafter, also referred to as PIP) that has a specific structure.

### BACKGROUND ART

The cornea is a highly-structured transparent tissue located in the anterior part of the eye which has no blood vessels. The cornea needs to be kept transparent to reflect light properly. An eye injury caused by an alkali burn is an eye surface disease that is severer than a thermal burn or a trauma caused by an acid and has poor prognosis. Alkali substances penetrate tissues very deeply and thereby cause serious clinical problems in the cornea, such as cloudiness in the cornea, which should naturally be transparent. Furthermore, since the injury is not adequately healed by conventional treatment, serious permanent impairment of visual function can occur. An alkali burn is one of major causes of acquired blindness.

The activation of corneal cells such as corneal keratocytes and epithelial cells and the influx of inflammatory cells such as mononuclear cells and macrophages during alkali burn are involved in the formation of a corneal injury lesion after the tissue is damaged by alkali, and can lead to permanent loss of the epithelium.

Acceleration of the healing process and prevention of formation of a scar tissue are two important clinical considerations after corneal injury. Many growth factors and cytokines are thought to be involved in tissue destruction and later formation of a scar that occur in the cornea after an alkali burn.

Furthermore, a damage such as a cloudy cornea may also occur after a corneal surgery (for example, LASIK) is performed to correct visual acuity or the like or after inflammation occurs due to an infection. The same growth factors and cytokines as involved in a trauma caused by an alkali burn appear to be involved in the healing process of inflammation such as a trauma after a corneal surgery.

The wound healing process is induced by transforming growth factor β (hereinafter, also referred to as “TGF-β”), which is secreted after an alkali burn occurs. Matrix metalloprotease (hereinafter, also referred to as “MMP”), which degrades the basement membrane, is secreted by the stimulus of TGF-β. This further promotes the defense mechanism against an infection or an external stimulus, such as induction of fibroblasts and angiogenesis. Destruction of the corneal basement membrane appears to contribute to the development of ulceration or perforation of the corneal stroma and to prevent regeneration of the corneal epithelium. Conjunctivalization of the corneal surface associated with opacification due to loss of stem cells at the corneal margin and neovascularization in the corneal stroma all cause impairment of visual function in patients in the later healing phase (Non Patent Literature 1). Since infections are controlled with antibiotics or the like in clinical practice in recent years, more advantages are expected in wound healing if the basement membrane destruction, cell infiltration, or neovascularization does not occur.

Furthermore, it has been shown that TGF-β not only promotes migration of corneal epithelial cells and keratocytes, but also promotes chemotaxis of mononuclear cells and macrophages and induces differentiation of corneal keratocytes to myofibroblasts. Overexpression of TGF-β and MMP9 located downstream thereof in the cornea having an inflammatory reaction caused by an external stimulus such as a burn or an infection worsens damage of the affected tissue because TGF-β partially induces the expression of other cytokines such as vascular endothelial growth factor (hereinafter, also referred to as “VEGF”) and monocyte/macrophage chemotaxis protein-1 (hereinafter, also referred to as “MCP-1”), which appears to be involved in local neovascularization and inflammation, respectively.

Furthermore, not only a trauma due an alkali burn, proliferative ophthalmic diseases such as tumor, proliferative diabetic retinopathy, pterygium, neovascular glaucoma, age-related macular degeneration, and corneal neovascularization associated with a corneal disease are known as proliferative tissue diseases in the ophthalmologic field. Particularly with aging of the society, more patients in the ophthalmologic field have diseases associated with abnormal neovascularization or fibrosis in recent years. These diseases are a particularly problematic syndrome because patients with this syndrome have high risks of decreased visual acuity and loss of vision, need a surgical operation, and are likely to experience recurrence. Since similar cell infiltration and neovascularization occur in inflammatory diseases, the same onset mechanism is conceivable in infiltration of corneal inflammatory cells associated with an infection, keratitis, conjunctivitis, allergic disease, or pollinosis. The same growth factors and cytokines as involved in the healing process of a trauma due an alkali burn are thought to be involved in this group of diseases.

Many patients who have visual impairment undergo surgical transplantation of an autograft or an allograft of the epithelium at the corneal margin containing corneal epithelial stem cells. Such transplantation is effectively performed in emergency, for example, in such a case where both eyes have been alkali-injured. However, since immunosuppression with a drug is necessary over a long period, potential risks for infections increase, and other untoward adverse drug reactions occur. Furthermore, many of alkali injury cases are so serious that transplantation itself is difficult. Furthermore, many patients with visual impairment are indicated in and waiting for corneal transplant. In reality, one third of patients have to wait for corneal transplant for 6 months or longer even in a successful eye bank. A nationwide eye bank survey (2004) revealed that patients have to wait for transplant for 3 years or longer.

A drug therapy for an intraophthalmic disease requires a drug delivery system (hereinafter, also referred to as “DDS”) that delivers an active ingredient of a drug to a target tissue in the eye effectively. In most cases, a drug administered by instillation does not adequately penetrate through the cornea. A drop of the drug flows out with a lachrymal fluid or absorbed in the body through the conjunctiva. Only approximately 5% of the drug administered by instillation penetrates through the cornea and reaches the ocular tissue, and the remaining 95% is lost together with the discharge of tears. Therefore, frequent (every few hours) administration is required for usual drugs. However, instillation is practically impossible during the sleep. Development of an eye drop with a prolonged action is therefore needed.

Aptamer-based therapeutic agents for VEGF inhibition are already available as a method for treating age-related macular degeneration. However, these drugs have disadvantages that administration is not easy because the drugs need to be frequently injected into the eye by intravitreal administration owing to the above-described drug delivery problem, and that frequent administration increases risks of infections from intraocular injection. Since intravenous administration of a drug is systemic administration, the influence on the whole body is increased, and concerns about adverse drug reactions and the like are raised when the intraocular drug concentration is maintained. Therefore, development of a topical therapeutic agent for an ophthalmic disease that does not need to be administered frequently is also demanded.

A pyrrole-imidazole polyamide is a chemically synthesized substance discovered by Dervan et al. based on the finding that duocarmycin-A and distamycin-A, which are antibiotics, recognize DNA nucleotide-specifically (Patent Literature 1 and Non Patent Literatures 2 and 3). Since the PI polyamide recognizes double-stranded DNA nucleotide sequence-specifically and binds to a minor groove of the DNA double helix structure, expression of a target gene can be specifically regulated (Non Patent Literature 3). Furthermore, unlike conventional gene expression regulating agents using antisense, ribozymes, siRNA, or the like, the PI polyamide is not degraded by nucleolytic enzymes in the organism and has a high ability to bind to a nucleic acid, and clinical application thereof to an anticancer agent or the like is expected as a novel molecularly-targeted therapeutic agent. However, no investigation has been performed for indication for ophthalmic diseases, instillation, or kinetics as a drug in the eyeball.

A technique for inactivating a gene function by reverse genetics is used to analyze a function of a specific gene and has a high potential for treatment of virus infection, cancer, and other diseases caused by abnormal gene expression. Specifically, it is known that a gene function can be inactivated by homologous recombination at a DNA level or at an RNA level using an antisense oligodeoxynucleotides or a ribozyme. However, homologous recombination generally has a low recombination rate and has problems that effects are seen in only some cells, technique using an antisense oligodeoxynucleotide or a ribozyme have limitation to targeted sequences, penetration into tissues or cells is poor, and RNA are easily degraded by ribonucleases.

Meanwhile, it has been reported that, unlike (deoxy)ribonucleotide reagents such as antisense reagents and ribozymes, pyrrole-imidazole polyamides specifically recognize nucleotide sequences of DNA and can control expression of a specific gene extracellularly.

A pyrrole-imidazole polyamide (hereinafter, also referred to as a Py-Im polyamide) is a group of small synthetic molecules: an N-methylpyrrole unit (hereinafter, also referred to as Py), which is an aromatic ring, and an N-methylimidazole unit (hereinafter, also referred to as Im) (Patent Literature 1 and Non Patent Literature 1). Py and Im can be sequentially coupled and folded to form a U-shaped conformation in the presence of γ-aminobutyric acid. In the pyrrole-imidazole polyamide according to the present invention, an N-methylpyrrole unit (Py), an N-methylimidazole unit (Im), β-alanine (β), and a γ-aminobutyric acid unit (hereinafter, also referred to as a γ-linker) are linked to each other with an amide bond (—C(═O)—NH—). General structures and production methods thereof are known (Patent Literatures 2 to 4).

Such a synthetic polyamide can bind to a specific base pair in a small groove (minor groove) of double helical DNA with high affinity and specificity. The recognition of a specific base pair depends on one-to-one pair formation of Py and Im. Specifically, in the U-shaped conformation in a minor groove of DNA, a Py/Im or β/Im pair targets a C-G base pair, Im/Py or Im/β targets a G-C base pair, and Py/Py, β/β, Py/β, or β/Py targets both an A-T base pair and a T-A base pair (Non Patent Literatures 2, 3, 4, and 5). According to recent researches, it has been found out that the problem of nonselectivity of the Py-Im polyamide compound for an A-T pair and a T-A pair can be solved by allowing a 3-hydroxypyrrole (Hp)/Py pair, which is obtained by substituting one pyrrole ring of a Py/Py pair with Hp, to bind preferentially to a T/A pair.

Furthermore, preferably, a part of a C-G base pair may be targeted by a β/Im pair, a part of a G-C base pair may be targeted by an Im/β pair, and parts of an A-T base pair and a T-A base pair may be targeted by a β/β pair, a Py/β pair, or a β/Py pair.

Generally, the initiation of transcription is thought to be a critical point of gene regulation. To initiate transcription, a transcription factor binding to a specific recognition sequence in the gene promoter region forms a complex, and the complex requires several steps of binding to a DNA sequence. If the binding of a transcription factor or a complex to a specific sequence is important in gene expression, a polyamide in a minor groove can block the binding of the transcription factor or a complex thereof and interfere with gene regulation. This hypothesis has been proved in vitro and in vivo. The 8-membered ring Py-Im polyamide binding to the inside of a recognition site (binding site of TFIIIA) of a zinc finger inhibited transcription of the 5S RNA gene. Polyamides binding to a base pair adjacent to a transcription factor sequence in the human immunodeficiency virus type 1 (HIV-1) promoter inhibit replication of HIV-1 in human cells. These sequences contain the TATA box, lymphatic system enhancer factor (LEF-1) sequence, and the ETS-1 sequence. On the other hand, polyamides activate a gene expression by blocking a repressor factor or substituting a naturally occurring transcription factor. The human cytomegalovirus (CMV) UL 122-mediated early protein 2 (IE86) blocks the supply of RNA polymerase II to a promoter, and inhibits transcription of related genes of CMV. Synthetic polyamides can block the inhibitions by IE86 and allow expression of the related genes. The polyamide designed by Mapp et al. acts as an artificial transcription factor and mediates a gene transcription reaction. Furthermore, it has been reported that a polyamide compound designed to bind to a site 10 nucleotides away from the binding sequence showed competitive inhibition in an experiment of inhibition of binding of a TATA box binding protein (TBP) to the TATA box, and inhibition of transcription, demonstrating that this inhibition of binding of a transcription factor is also achieved by a polyamide designed to bind to a region surrounding a transcription factor recognition sequence (Non Patent Literature 6). Furthermore, the rat MMP9AP1 polyamide described in the present application is designed to be located 4 nucleotides closer to the transcription initiation point from the AP1-binding sequence, but it can be easily imagined that inhibition of binding to AP1 is induced.

### Citation List

### Patent Literature

Patent Literature 1: WO/1998/49142 A1
Patent Literature 2: Japanese Patent No. 3045706
Patent Literature 3: JP 2001-136974 A
Patent Literature 4: WO/2003/000683 A1
Patent Literature 5: WO/2006/018967 A1

### Non Patent Literature

Non Patent Literature 1: Saika S. et al. American Journal of Pathology 2005; 166(5): 1405-1418.
Non Patent Literature 2: Sugiyama et al. Proc Natl Acad Sci USA. 1996; 93: 14405-144410.
Non Patent Literature 3: Dervan: Bioorg Med. Chem. 2001; 9: 215-35.
Non Patent Literature 4: Trauger J. W. et al. Nature 1996; 382: 559-561.
Non Patent Literature 5: White S. et al. Chem Biol 1997; 4: 569-578.
Non Patent Literature 6: Ehley J. A. et al. Mol Cell Biol 2002; 22(6): 1723-1733.

### SUMMARY OF INVENTION

### Technical Problem

So far, no topical therapeutic agents for an ophthalmic disease for the treatment of a trauma due an alkali burn or a trauma after a corneal surgery have been reported that are not administered by intraocular injection and are instilled or the like. Furthermore, no topical therapeutic agents for an ophthalmic disease for the treatment of proliferative ophthalmic diseases have been reported that do not have to be administered frequently.

Accordingly, a topical therapeutic agent for an ophthalmic disease for the treatment of a trauma due an alkali burn, a trauma after a corneal surgery, or a proliferative ophthalmic disease is demanded.

### Solution to Problem

The present inventors found that a pyrrole-imidazole polyamide that can inhibit the expression of the transforming growth factor β (TGF-β) gene and the matrix metalloprotease 9 (MMP9) gene by specifically binding to specific regions of the promoters of transforming growth factor β and matrix metalloprotease 9 can effectively act as a topical therapeutic agent for an ophthalmic disease, and thus accomplished the present invention.

Specifically, the present invention is as follows.

(1) A topical therapeutic agent for an ophthalmic disease containing a pyrrole-imidazole polyamide comprising an N-methylpyrrole unit (hereinafter, also referred to as Py), an N-methylimidazole unit (hereinafter, also referred to as Im), and a γ-aminobutyric acid unit, wherein the pyrrole-imidazole polyamide can be folded at the γ-aminobutyric acid unit to form a U-shaped conformation in a minor groove of a double helical region (hereinafter, also referred to as a target region) which comprises a part or a whole of a nucleotide sequence from −555 to −528 (SEQ ID NO: 2), a nucleotide sequence from −427 to −399 (SEQ ID NO: 4), or a nucleotide sequence from −384 to −355 (SEQ ID NO: 6) in a human transforming growth factor (hereinafter, also referred to as TGF-β) gene promoter, and a strand complementary thereto, wherein a Py/Im pair corresponds to a C-G base pair, an Im/Py pair corresponds to a G-C base pair, and a Py/Py pair corresponds to both an A-T base pair and a T-A base pair.
(2) The topical therapeutic agent for an ophthalmic disease according to (1), further comprising a β-alanine unit.
(3) The topical therapeutic agent for an ophthalmic disease according to (1) or (2), further comprising a fluorescein isothiocyanate unit.
(4) The topical therapeutic agent for an ophthalmic disease according to any one of (1) to (3), wherein the ophthalmic disease is a trauma due to an alkali burn or a trauma after a corneal surgery.
(5) The topical therapeutic agent for an ophthalmic disease according to any one of (1) to (3), wherein the ophthalmic disease is a proliferative ophthalmic disease or an inflammatory ophthalmic disease.
(6) The topical therapeutic agent for an ophthalmic disease according to any one of (1) to (5), which is in the form of an eye drop.
(7) The topical therapeutic agent for an ophthalmic disease according to any one of (1) to (6), wherein the target region is a double helical region which comprises a part or a whole of a nucleotide sequence from −544 to −538 (SEQ ID NO: 3), a nucleotide sequence from −416 to −410 (SEQ ID NO: 5), or a nucleotide sequence from −373 to −366 (SEQ ID NO: 7) in the transforming growth factor β promoter, and a strand complementary thereto.
(8) The topical therapeutic agent for an ophthalmic disease according to any one of (1) to (7), wherein the pyrrole-imidazole polyamide is represented by the following formula:

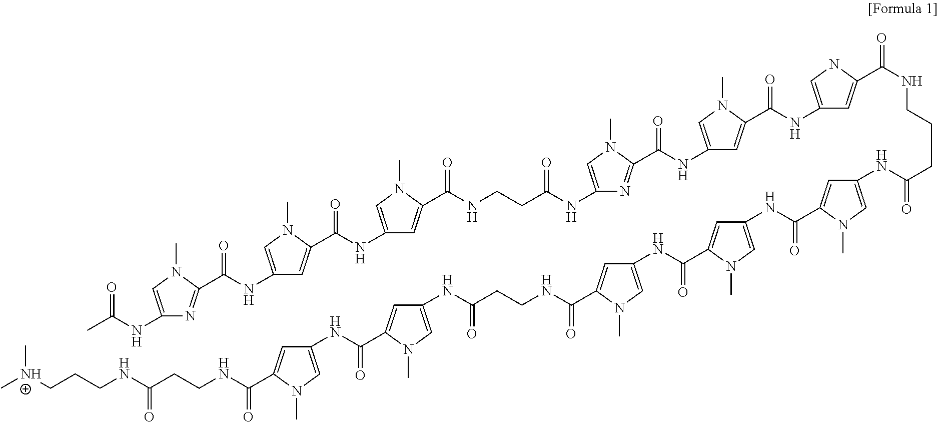

(9) The topical therapeutic agent for an ophthalmic disease according to any one of (1) to (7), wherein the pyrrole-imidazole polyamide is represented by the following formula:

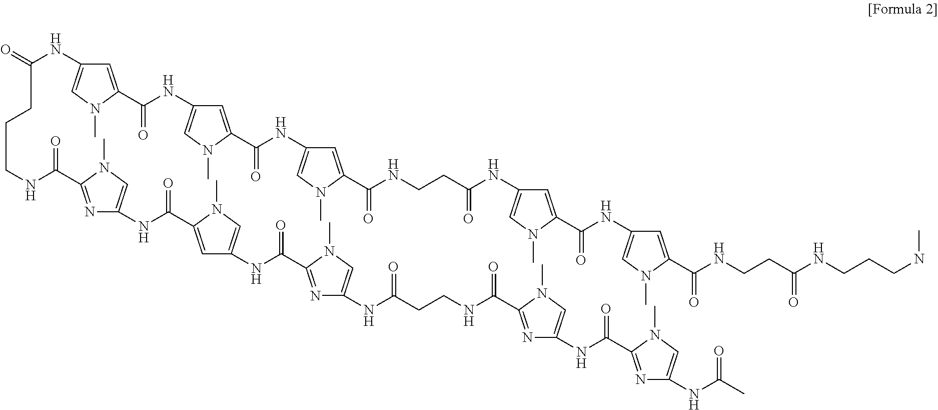

(10) The topical therapeutic agent for an ophthalmic disease according to any one of (1) to (7), wherein the pyrrole-imidazole polyamide is represented by the following formula:

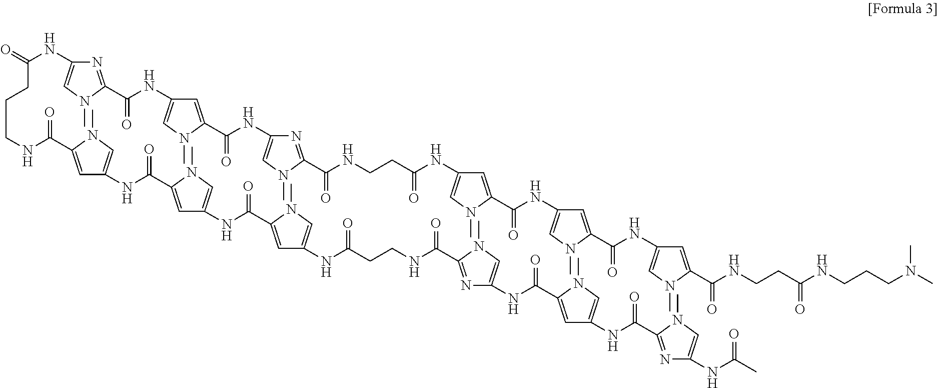

(11) A topical therapeutic agent for an ophthalmic disease containing a pyrrole-imidazole polyamide represented by the following formula:

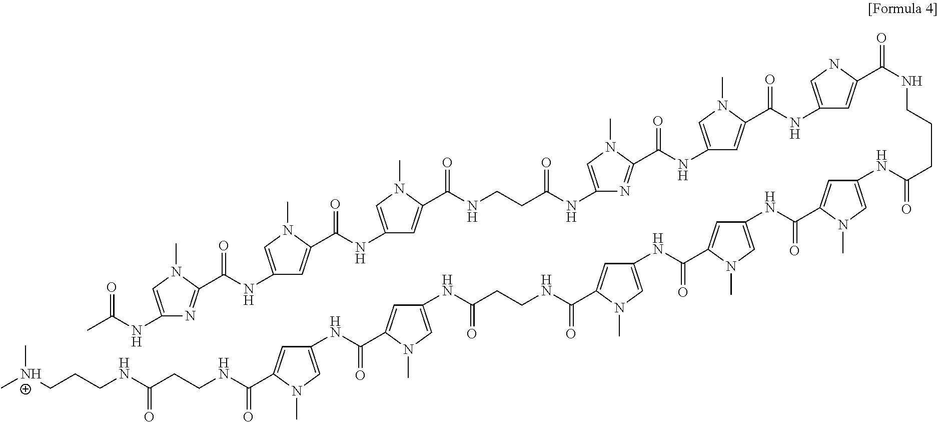

(12) A topical therapeutic agent for an ophthalmic disease containing a pyrrole-imidazole polyamide represented by the following formula:

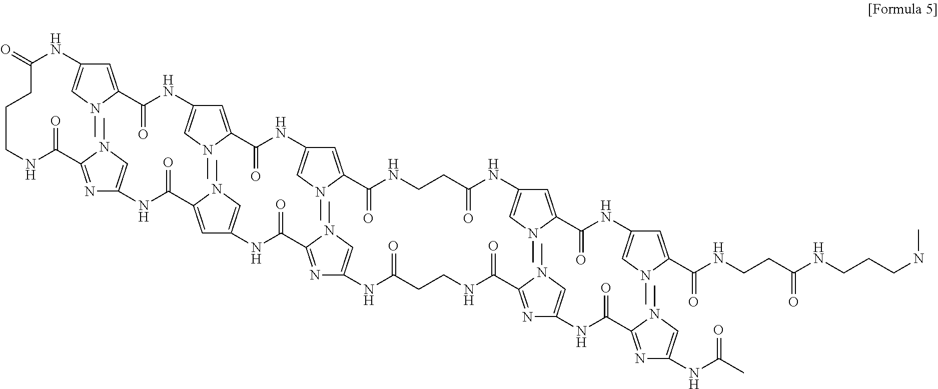

(13) A topical therapeutic agent for an ophthalmic disease containing a pyrrole-imidazole polyamide represented by the following formula:

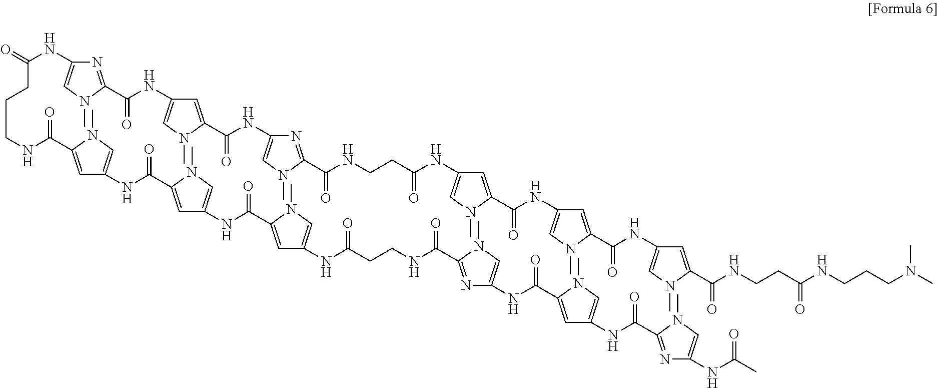

(14) A topical therapeutic agent for an ophthalmic disease containing a pyrrole-imidazole polyamide comprising an N-methylpyrrole unit (hereinafter, also referred to as Py), an N-methylimidazole unit (hereinafter, also referred to as Im), and a γ-aminobutyric acid unit, wherein the pyrrole-imidazole polyamide can be folded at the γ-aminobutyric acid unit to form a U-shaped conformation in a minor groove of a double helical region (hereinafter, also referred to as a target region) which comprises a part or a whole of a nucleotide sequence from −2316 to −2287 (SEQ ID NO: 25) or a nucleotide sequence from −2322 to −2293 (SEQ ID NO: 8) in a rat transforming growth factor β (hereinafter, also referred to as TGF-β) gene promoter, and a strand complementary thereto, wherein a Py/Im pair corresponds to a C-G base pair, an Im/Py pair corresponds to a G-C base pair, and a Py/Py pair corresponds to both an A-T base pair and a T-A base pair.
(15) The topical therapeutic agent for an ophthalmic disease according to (14), further comprising a β-alanine unit.
(16) The topical therapeutic agent for an ophthalmic disease according to (14) or (15), further comprising a fluorescein isothiocyanate unit.
(17) The topical therapeutic agent for an ophthalmic disease according to any one of (14) to (16), wherein the ophthalmic disease is a trauma due to an alkali burn or a trauma after a corneal surgery.
(18) The topical therapeutic agent for an ophthalmic disease according to any one of (14) to (16), wherein the ophthalmic disease is a proliferative ophthalmic disease or an inflammatory ophthalmic disease.
(19) The topical therapeutic agent for an ophthalmic disease according to any one of (14) to (18), which is in the form of an eye drop.
(20) The topical therapeutic agent for an ophthalmic disease according to any one of (14) to (19), wherein the target region is a double helical region which comprises a part or a whole of a nucleotide sequence from −2305 to −2298 (SEQ ID NO: 26) or a nucleotide sequence from −2311 to −2304 (SEQ ID NO: 9) in the transforming growth factor β promoter, and a strand complementary thereto.
(21) The topical therapeutic agent for an ophthalmic disease according to any one of claims (14) to (20), wherein the pyrrole-imidazole polyamide is represented by the following formula:

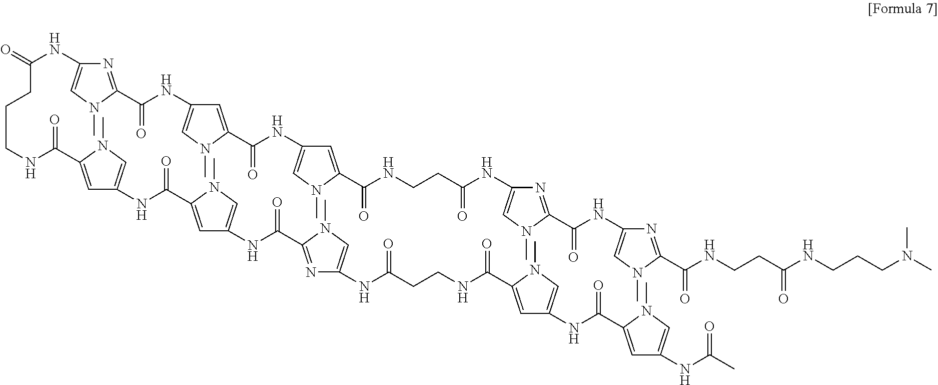

(22) The topical therapeutic agent for an ophthalmic disease according to any one of (14) to (20), wherein the pyrrole-imidazole polyamide is represented by the following formula:

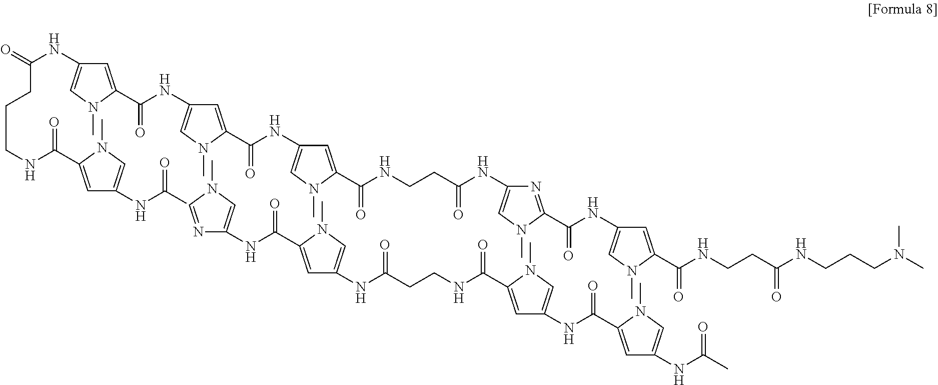

(23) A topical therapeutic agent for an ophthalmic disease containing a pyrrole-imidazole polyamide represented by the following formula:

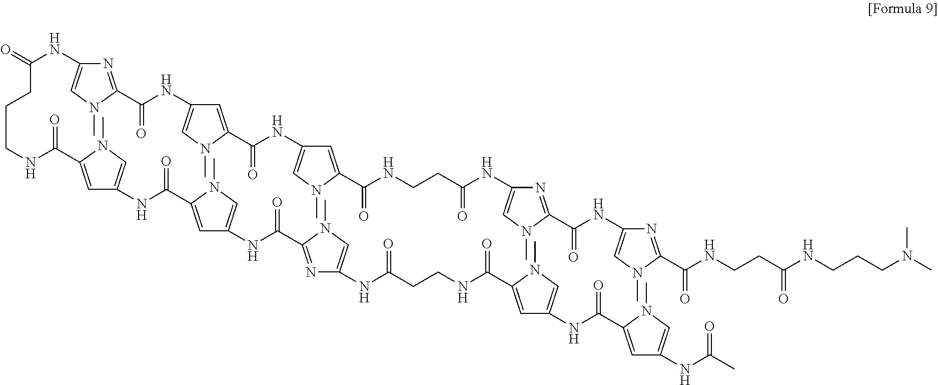

(24) A topical therapeutic agent for an ophthalmic disease containing a pyrrole-imidazole polyamide represented by the following formula:

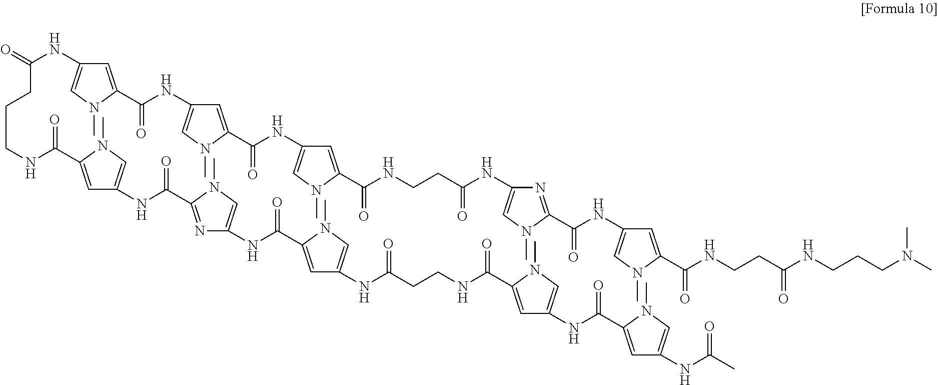

(25) A topical therapeutic agent for an ophthalmic disease containing a pyrrole-imidazole polyamide comprising an N-methylpyrrole unit (hereinafter, also referred to as Py), an N-methylimidazole unit (hereinafter, also referred to as Im), and a γ-aminobutyric acid unit, wherein the pyrrole-imidazole polyamide can be folded at the γ-aminobutyric acid unit to form a U-shaped conformation in a minor groove of a double helical region (hereinafter, also referred to as a target region) which comprises a part or a whole of a nucleotide sequence from −88 to −59 (SEQ ID NO: 11) or a nucleotide sequence from −616 to −588 (SEQ ID NO: 13) in a human matrix metalloprotease 9 (hereinafter, also referred to as hMMP-9) gene promoter, and a strand complementary thereto, wherein a Py/Im pair corresponds to a C-G base pair, an Im/Py pair corresponds to a G-C base pair, and a Py/Py pair corresponds to both an A-T base pair and a T-A base pair.
(26) The topical therapeutic agent for an ophthalmic disease according to (25), further comprising a β-alanine unit.
(27) The topical therapeutic agent for an ophthalmic disease according to (25) or (26), further comprising a fluorescein isothiocyanate unit.
(28) The topical therapeutic agent for an ophthalmic disease according to any one of (25) to (27), wherein the ophthalmic disease is a trauma due to an alkali burn or a trauma after a corneal surgery.
(29) The topical therapeutic agent for an ophthalmic disease according to any one of (25) to (27), wherein the ophthalmic disease is a proliferative ophthalmic disease or an inflammatory ophthalmic disease.
(30) The topical therapeutic agent for an ophthalmic disease according to any one of (25) to (29), which is in the form of an eye drop.
(31) The topical therapeutic agent for an ophthalmic disease according to any one of (25) to (30), wherein the target region is a double helical region which comprises a part or a whole of a nucleotide sequence from −77 to −70 (SEQ ID NO: 12) or a nucleotide sequence from −605 to −599 (SEQ ID NO: 14) in a transforming growth factor β promoter, and a strand complementary thereto.
(32) The topical therapeutic agent for an ophthalmic disease according to any one of (25) to (31), wherein the pyrrole-imidazole polyamide is represented by the following formula:

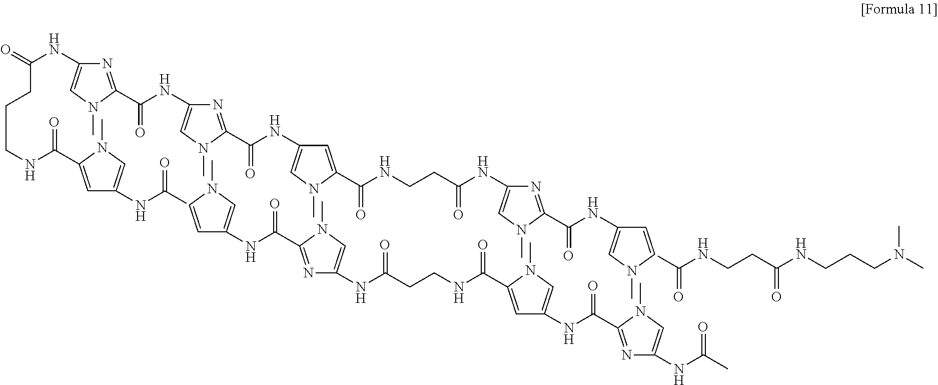

(33) The topical therapeutic agent for an ophthalmic disease according to any one of (25) to (31), wherein the pyrrole-imidazole polyamide is represented by the following formula:

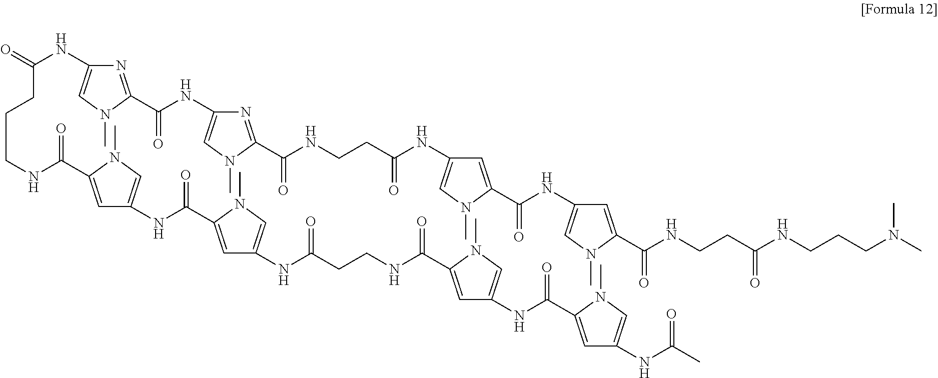

(34) A topical therapeutic agent for an ophthalmic disease containing a pyrrole-imidazole polyamide represented by the following formula:

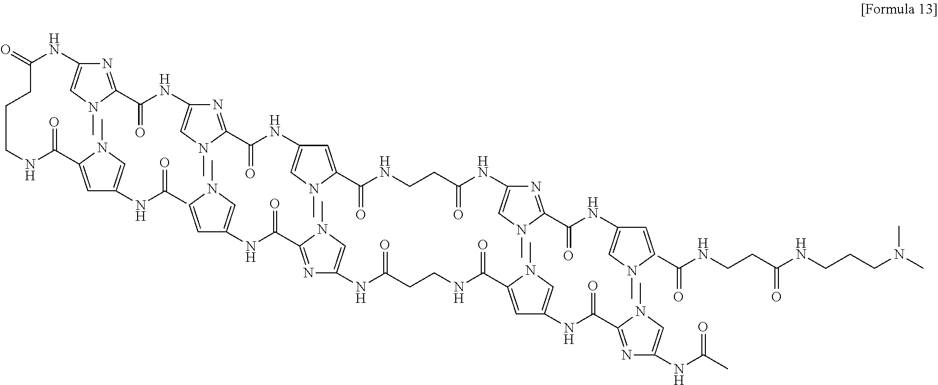

(35) A topical therapeutic agent for an ophthalmic disease containing a pyrrole-imidazole polyamide represented by the following formula:

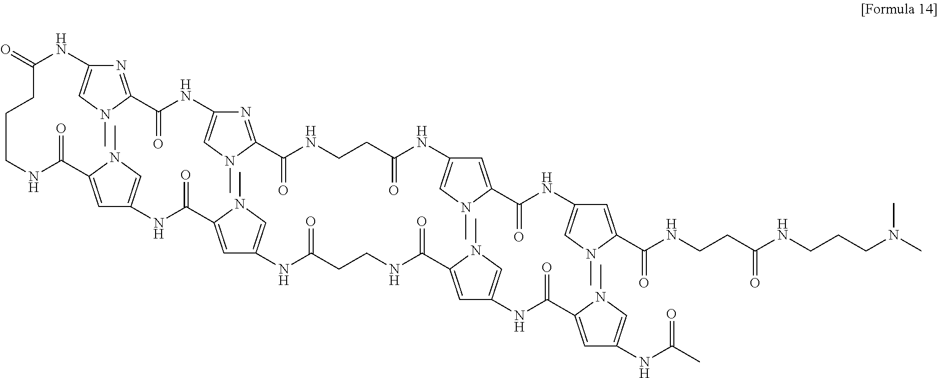

(36) A topical therapeutic agent for an ophthalmic disease containing a pyrrole-imidazole polyamide comprising an N-methylpyrrole unit (hereinafter, also referred to as Py), an N-methylimidazole unit (hereinafter, also referred to as Im), and a γ-aminobutyric acid unit, wherein the pyrrole-imidazole polyamide can be folded at the γ-aminobutyric acid unit to form a U-shaped conformation in a minor groove of a double helical region (hereinafter, also referred to as a target region) which comprises a part or a whole of a nucleotide sequence from −105 to −76 (SEQ ID NO: 15) or a nucleotide sequence from −602 to −575 (SEQ ID NO: 17) in a rat matrix metalloprotease 9 (hereinafter, also referred to as rMMP-9) gene promoter, and a strand complementary thereto, wherein a Py/Im pair corresponds to a C-G base pair, an Im/Py pair corresponds to a G-C base pair, and a Py/Py pair corresponds to both an A-T base pair and a T-A base pair.
(37) The topical therapeutic agent for an ophthalmic disease according to (36), further comprising a β-alanine unit.
(38) The topical therapeutic agent for an ophthalmic disease according to (36) or (37), further comprising a fluorescein isothiocyanate unit.
(39) The topical therapeutic agent for an ophthalmic disease according to any one of (36) to (38), wherein the ophthalmic disease is a trauma due to an alkali burn or a trauma after a corneal surgery.
(40) The topical therapeutic agent for an ophthalmic disease according to any one of (36) to (38), wherein the ophthalmic disease is a proliferative ophthalmic disease or an inflammatory ophthalmic disease.
(41) The topical therapeutic agent for an ophthalmic disease according to any one of (36) to (40), which is in the form of an eye drop.
(42) The topical therapeutic agent for an ophthalmic disease according to any one of (36) to (41), wherein the target region is a double helical region which comprises a part or a whole of a nucleotide sequence from −94 to −87 (SEQ ID NO: 16) or a nucleotide sequence from −591 to −586 (SEQ ID NO: 18) in the matrix metalloprotease 9 promoter, and a strand complementary thereto.
(43) The topical therapeutic agent for an ophthalmic disease according to any one of (36) to (42), wherein the pyrrole-imidazole polyamide is represented by the following formula:

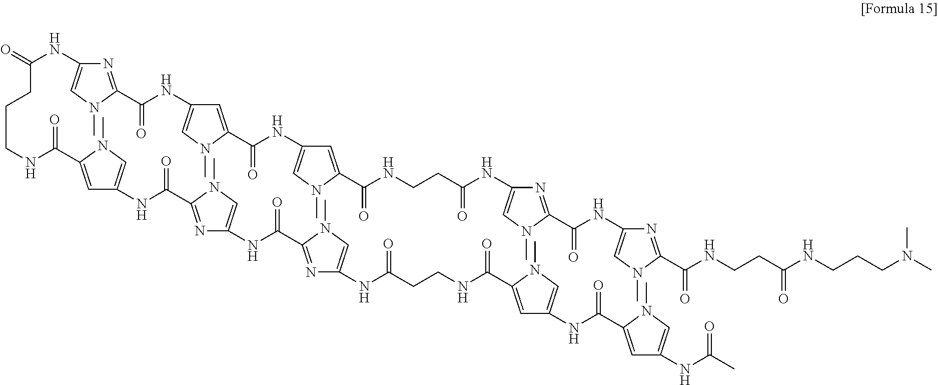

(44) The topical therapeutic agent for an ophthalmic disease according to any one of (36) to (42), wherein the pyrrole-imidazole polyamide is represented by the following formula:

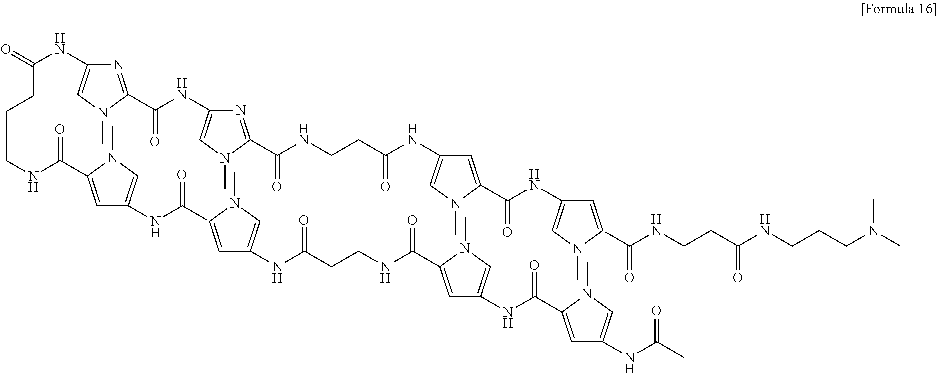

(45) A topical therapeutic agent for an ophthalmic disease containing a pyrrole-imidazole polyamide represented by the following formula:

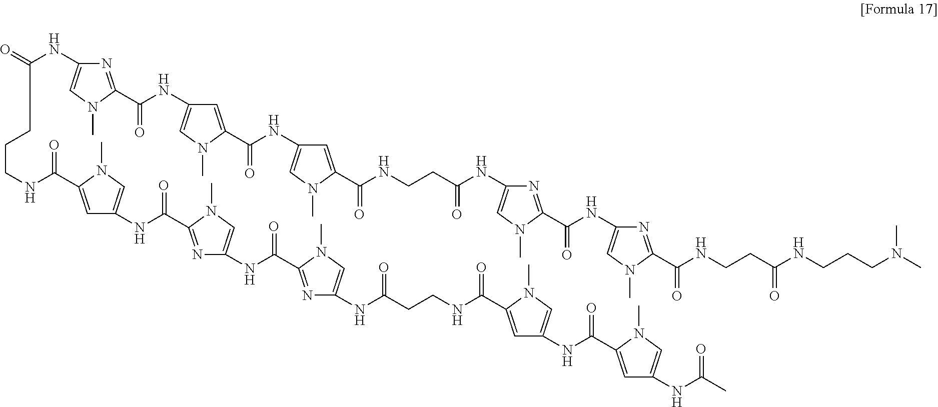

(46) A topical therapeutic agent for an ophthalmic disease containing a pyrrole-imidazole polyamide represented by the following formula:

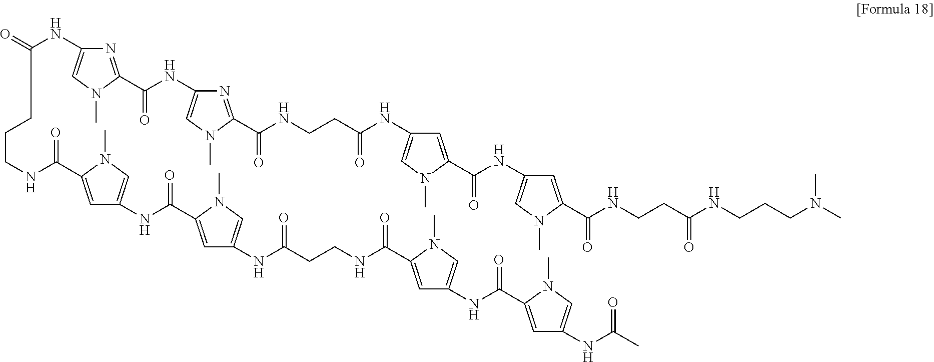

### Advantageous Effects of Invention

According to the present invention, a topical therapeutic agent for an ophthalmic disease for the treatment of a trauma due an alkali burn, a trauma after a corneal surgery, a proliferative ophthalmic disease, or an inflammatory ophthalmic disease can be obtained. Furthermore, according to the present invention, a reagent for a basic experiment using genes can be obtained.

### BRIEF DESCRIPTION OF DRAWINGS

FIG. 1 shows the nucleotide sequence of the human TGF-β promoter region.

FIG. 2 shows the rTGF-β (GBP1201) PIP compound binding site in the rat TGF-β promoter region.

FIG. 3 shows the rTGF-β (GBP1203) PIP compound binding site in the rat TGF-β promoter region.

FIG. 4 shows the rMMP9AP1 PIP compound binding site in the rat MMP-9 promoter region.

FIG. 5 shows the rMMP9NFκB PIP compound binding site in the rat MMP-9 promoter region.

FIG. 6 shows the nucleotide sequence of the human MMP-9 promoter region.

FIG. 7 shows the hMIMP9AP1 PIP compound binding site in the human MMP-9 promoter region.

FIG. 8 shows the hMMP9NFκB PIP compound binding site in the human MMP-9 promoter region.

FIG. 9 shows a PIP compound (hTGF-β [GBP1101]) binding to a nucleotide sequence from −544 to −538 in the human TGF-β promoter.

FIG. 10 shows a PIP compound (hTGF-β [GBP1105]) binding to a nucleotide sequence from −416 to −410 in the human TGF-β promoter.

FIG. 11 shows a PIP compound (hTGF-β [GBP1106]) binding to a nucleotide sequence from −373 to −366 in the human TGF-β promoter.

FIG. 12 shows a PIP compound (rTGF-β [GBP1201]) binding to a nucleotide sequence from −2311 to −2304 in the rat TGF-β promoter.

FIG. 13 shows a PIP compound (rTGF-β [GBP1203]) binding to a nucleotide sequence from −2305 to −2298 in the rat TGF-β promoter.

FIG. 14 shows a PIP compound (hMMP9AP1) binding to a nucleotide sequence from −77 to −70 in the human MMP-9 (AP-1) promoter.

FIG. 15 shows a PIP compound (hMMP9NFκB) binding to a nucleotide sequence from −605 to −599 in the human MMP-9 (NF-κB) promoter.

FIG. 16 shows a PIP compound (rMMP9AP1) binding to a nucleotide sequence from −94 to −87 in the rat MMP-9(AP-1) promoter.

FIG. 17 shows a PIP compound (rMMP9NFκB) binding to a nucleotide sequence from −591 to −586 in the rat MMP-9 (NF-κB) promoter.

FIG. 18 shows effects of the PIP compound of the present invention in the rat model after an alkali injury.

FIG. 19 shows the results of evaluation of corneal opacity and ulceration. rTGF-β1 (GBP1201) PIP and MMP-9 PIP healed corneal opacity and ulceration significantly better than the control.

FIG. 20 shows the results of evaluation of corneal opacity and ulceration. rTGF-β1 (GBP1201) PIP healed corneal opacity and ulceration significantly better than the control.

FIG. 21 shows the results of evaluation of severity of a corneal defect. MMP-9 PIP and TGF-β (GBP1201) PIP healed the corneal defect significantly earlier (approximately twice as fast) than the control.

FIG. 22 shows the results of evaluation of corneal opacity and ulceration. rTGF-β1 (GBP1201) PIP healed corneal opacity and ulceration significantly better than the control.

FIG. 23 shows the results of a real time RT-PCR assay for determining expression levels of TGF-β mRNA by rTGF-β (GBP1201).

FIG. 24 shows the results of a real time RT-PCR assay for determining expression levels of MMP-9 mRNA by rTGF-β1 (GBP1201).

FIG. 25 shows the results of a real time RT-PCR assay for determining expression levels of TGF-β mRNA by rTGF-β1 (GBP1201).

FIG. 26 shows the results of a real time RT-PCR assay for determining expression levels of MMP-9 mRNA by rMMP9AP1.

FIG. 27 shows a PIP compound (hereinafter, also referred to as rTGF-β [GBP1201] FITC) binding to a nucleotide sequence from −2311 to −2304 in the FITC-labeled rat TGF-β promoter.

FIG. 28 shows a mismatch polyamide against a nucleotide sequence from −2311 to −2304 in the rat TGF-β promoter.

FIG. 29 shows a PIP compound (hereinafter, also referred to as rMMP9AP1FITC) binding to a nucleotide sequence from −94 to −87 in the FITC-labeled rat MMP-9(AP-1) promoter.

FIG. 30 shows the distributions of the FITC-labeled rat rTGF-β (GBP1201) PIP compound administered after an alkali injury in the cornea at 1 hour and on days 1, 4, and 7.

FIG. 31 shows the distribution of the FITC-labeled rat rTGF-β (GBP1201) PIP compound administered after an alkali injury in the cornea at 1 hour.

FIG. 32 shows the results of an immunohistochemistry test of rat eyes using rTGF-β1 (GBP1201) PIP.

FIG. 33 shows a total ion chromatogram (TIC) chart and electrospray ionization mass spectrometry spectra of the PIP compounds hTGF-β (GBP1105) and (GB1106) of the present invention.

FIG. 34 shows a total ion chromatogram (TIC) chart and electrospray ionization mass spectrometry spectra of the PIP compounds hTGF-β (GBP1101) of the present invention.

FIG. 35a shows RP-HPLC charts of the PIP compounds rTGF-β (GBP1201) and rMMP9AP1 FITC of the present invention in (a) and (b).

FIG. 35b shows RP-HPLC charts of the PIP compounds rMMP9AP1 and rTGF-β (GBP1201) FITC of the present invention in (a) and (b).

FIG. 36 shows an RP-HPLC chart of the PIP compound rMMPNFκβ of the present invention.

FIG. 37 shows an RP-HPLC chart of the PIP compound hMMP9AP1 of the present invention.

FIG. 38 shows an RP-HPLC chart of the PIP compound hMMP9NFκβ of the present invention.

FIG. 39 shows an RP-HPLC chart of the PIP compound rTGF-β (GBP1201) of the present invention.

FIG. 40 shows the results of investigation of specificity and affinity of binding of rTGF-β (GBP1201) PIP to a target DNA. (A) shows the results of a gel shift assay. Lane 1 shows the result of a single-stranded DNA, lane 2 shows the result of a double-stranded DNA, and lane 3 shows the result of rTGF-β (GBP1201) and double-stranded DNA. (B) shows the results of a BIACORE assay. The graph shows the results of rTGF-β (GBP1201) at concentrations of 0, 1, 5, 10, 20, 30, 50, 75, 100, 150, 200, and 300 nM from the bottom. (C) shows the results of a BIACORE assay. The graph shows the results of a mismatch polyamide at concentrations of 0, 1, 5, 10, 20, 30, 50, 75, 100, 150, 200, and 300 nM. (D) shows the results of an assay for determining inhibition of the rTGF-β1 promoter expression in the promoter using a luciferase reporter plasmid in which the promoter region of rTGF-β1 was subcloned. The reporter gene expression induced by adding PMA was inhibited by adding GBP1201 and GBP1203 but not by the mismatch polyamide.

FIG. 41 shows structural formula (a) and an RP-HPLC chart (b) of the mismatch polyamide against the PIP compound of the present invention used in the experiment shown in FIG. 40.

FIG. 42 shows an RP-HPLC chart of rTGF-β1 (GBP1203), the PIP compound of the present invention.

FIG. 43 shows intracellular distributions and (intranuclear) localization of the FITC-labeled human hTGF-β1 (GBP1105) PIP compound of the present invention in human retina epithelial cell APRE-19 at 30 minutes and 2 and 6 hours after addition.

### DESCRIPTION OF EMBODIMENTS

In the pyrrole-imidazole polyamide according to the present invention, an N-methylpyrrole unit, an N-methylimidazole unit, and a γ-aminobutyric acid unit (hereinafter, also referred to as a γ linker) are linked to each other with an amide bond (—C(═O)—NH—), and general structures and production methods thereof are known (for example, refer to Patent Literatures 1 to 3).

For example, a pyrrole-imidazole polyamide can be produced by automated synthesis by a solid phase method using 9-fluorenylmethoxycarbonyl (Fmoc), the solid phase Fmoc method (Patent Literature 3). Since the end of a pyrrole-imidazole polyamide can be removed as a carboxylic acid residue from a solid carrier according to the solid phase Fmoc method, various functional groups can be introduced to a molecular end to prepare pyrrole-imidazole polyamide derivatives. For example, duocarmycin, pyrrolobenzodiazepine, bleomycin, an enediyne compound, a nitrogen mustard, or a derivative thereof, or a compound having an ability of alkylating DNA can also be introduced if necessary. Since the solid phase Fmoc method is an automated synthesis method using a commercially available protein (peptide) synthesizer, a conjugate of a naturally occurring protein or an unnatural protein and a pyrrole-imidazole polyamide can be synthesized. Furthermore, since the Fmoc method is employed under less strict reaction conditions compared with the t-BOC method, organic compounds (compounds that have unstable functional groups under an acidic condition) other than proteins can also be introduced. For example, a conjugate of a pyrrole-imidazole polyamide and DNA or RNA (or a derivative thereof) can also be automatically synthesized.

According to the above-described known Fmoc method or the like, a pyrrole-imidazole polyamide having a carboxyl group at an end can be synthesized. Specific examples of such a pyrrole-imidazole polyamide include pyrrole-imidazole polyamides having a β-alanine residue (β-aminopropionic acid residue) or a γ-aminobutyric acid residue at an end. For example, the pyrrole-imidazole polyamides having a β-alanine residue or a γ-aminobutyric acid residue at an end can be synthesized by the solid phase Fmoc method using a solid phase carrier carrying aminopyrrolecarboxylic acid, aminoimidazolecarboxylic acid, β-alanine, or γ-aminobutyric acid with an amino group protected with an Fmoc group with a peptide synthesizer.

Specific examples of the aminopyrrolecarboxylic acid include 4-amino-2-pyrrolecarboxylic acid, 4-amino-1-methyl-2-pyrrolecarboxylic acid, 4-amino-1-ethyl-2-pyrrolecarboxylic acid, 4-amino-1-propyl-2-pyrrolecarboxylic acid, and 4-amino-1-butyl-2-pyrrolecarboxylic acid. Specific examples of the aminoimidazolecarboxylic acid include 4-amino-2-imidazolecarboxylic acid, 4-amino-1-methyl-2-imidazolecarboxylic acid, 4-amino-1-ethyl-2-imidazolecarboxylic acid, 4-amino-1-propyl-2-imidazolecarboxylic acid, and 4-amino-1-butyl-2-imidazolecarboxylic acid.

According to the solid phase Fmoc method, for example, a conjugate of a pyrrole-imidazole polyamide and fluorescein isothiocyanate (FITC) can also be synthesized. FITC has long been known as a fluorescence-labeling reagent for an antibody. An obtained conjugate can be used to demonstrate that the pyrrole-imidazole polyamide recognizes a specific DNA sequence.

The pyrrole-imidazole compound of the present invention contains a pyrrole-imidazole polyamide comprising an N-methylpyrrole unit (Py), an N-methylimidazole unit (Im) and a γ-aminobutyric acid unit, wherein the pyrrole-imidazole polyamide can be folded to form a U-shaped conformation at the γ-aminobutyric acid unit in a minor groove of a double helical region (hereinafter, also referred to as a target region) which comprises a part or a whole of a nucleotide sequence region from −555 to −528 (SEQ ID NO: 2), a region from −427 to −399 (SEQ ID NO: 4), or a region from −384 to −355 (SEQ ID NO: 6) in a human transforming growth factor β, a nucleotide sequence region from −2316 to −2287 (SEQ ID NO: 25) or a region from −2322 to −2293 (SEQ ID NO: 8) in a rat transforming growth factor β, a nucleotide sequence region from −88 to −59 (SEQ ID NO: 11) or a region from −616 to −588 (SEQ ID NO: 13) in a human matrix metalloprotease 9, or a nucleotide sequence region from −105 to −76 (SEQ ID NO: 15) or a region from −602 to −575 (SEQ ID NO: 17) in rat matrix metalloprotease 9, and a strand complementary thereto, wherein a Py/Im pair corresponds to a C-G base pair, an Im/Py pair corresponds to a G-C base pair, and a Py/Py pair corresponds to both an A-T base pair and a T-A base pair.

Usually, a helical DNA skeleton forms two types of grooves. The wide and deep groove is called a major groove, and the narrow and shallow groove is called a minor groove. Here, the above-mentioned pyrrole-imidazole polyamide has high affinity and specificity for a minor groove formed by a specific base pair and can bind thereto in an unconjugated manner.

In this binding, a Py/Im pair or a β/Im pair of the pyrrole-imidazole polyamide corresponds to a C-G base pair in the minor groove, an Im/Py pair or an Im/β pair corresponds to a G-C base pair, and a Py/Py pair, a β/β pair, a Py/β pair, or a β/Py pair corresponds to both an A-T base pair and a T-A base pair. Furthermore, the pyrrole-imidazole polyamide molecule is folded to form a U-shaped conformation at the site of the γ-aminobutyric acid unit in the molecule.

If a base pair in the minor groove does not correspond to a pair of Py and Im of the pyrrole-imidazole polyamide in the manner described above, binding of the minor groove and the pyrrole-imidazole polyamide is not adequate. A pyrrole-imidazole polyamide in which a base pair in a minor groove does not correspond to a Py-Im pair as described above is referred to as a mismatch or a mismatch polyamide in the present application.

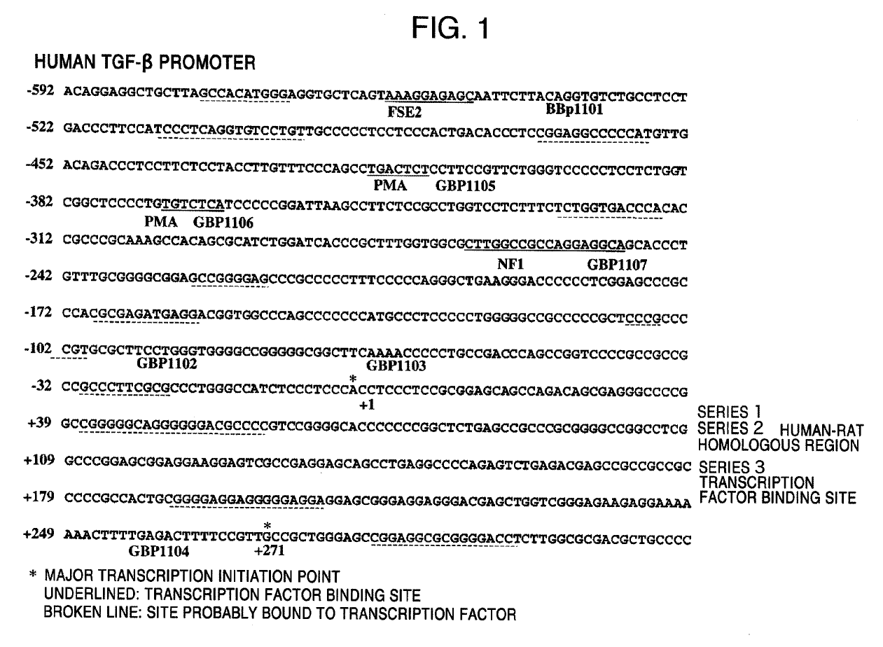

The nucleotide sequences of the regulatory regions of the human transforming growth factor β gene and the human matrix metalloprotease 9 gene are shown in FIGS. 1 (SEQ ID NO: 1) and 6 (SEQ ID NO: 10).

The human transforming growth factor β (GBP1101) (hereinafter, also referred to as hTGF-β [GBP1101]), the pyrrole-imidazole polyamide compound of the present invention, has a molecular formula of C₈₄H₁₀₃N₃₀O₁₆ and a molecular weight of 1788.91. By adding Dp to a polyamide compound described in Patent Literature 5 (WO/2006/018967) to add a positive electric charge, the structure has been improved to a structure with increased binding ability to DNA and water-solubility. The human transforming growth factor β (GBP1105) (hereinafter, also referred to as hTGF-β [GBP1105]) has a molecular formula of C₇₆H₉₄N₃₀O₁₅ and a molecular weight of 1667.75. The human transforming growth factor β (GBP1106) (hereinafter, also referred to as hTGF-β [GBP1106]) has a molecular formula of C₈₈H₁₀₆N₃₄O₁₇ and a molecular weight of 1912.00. In the hTGF-β gene regulatory region (SEQ ID NO: 1), the target sequence of GBP1101 is a fat specific element (FSE2)-binding region in the nucleotide sequence region of −555 to −528 (SEQ ID NO: 2), more specifically −544 to −538 (SEQ ID NO: 3), the target sequence of GBP1105 is the nucleotide sequence region of −427 to −399 (SEQ ID NO: 4) containing the AP1-binding sequence, more specifically −416 to −410 (SEQ ID NO: 5), and the target sequence of GBP1106 is the region of −384 to −355 (SEQ ID NO: 6) containing an AP1-binding sequence, more specifically −373 to −366 (SEQ ID NO: 7). Expression of the TGF-β1 gene is inhibited by allowing these pyrrole-imidazole polyamide compounds to bind to the vicinity of the transcription factor binding site.

The pyrrole-imidazole polyamides hTGF-β (GBP1101), hTGF-β (GBP1105), and hTGF-β (GBP1106) of the present invention are shown below.

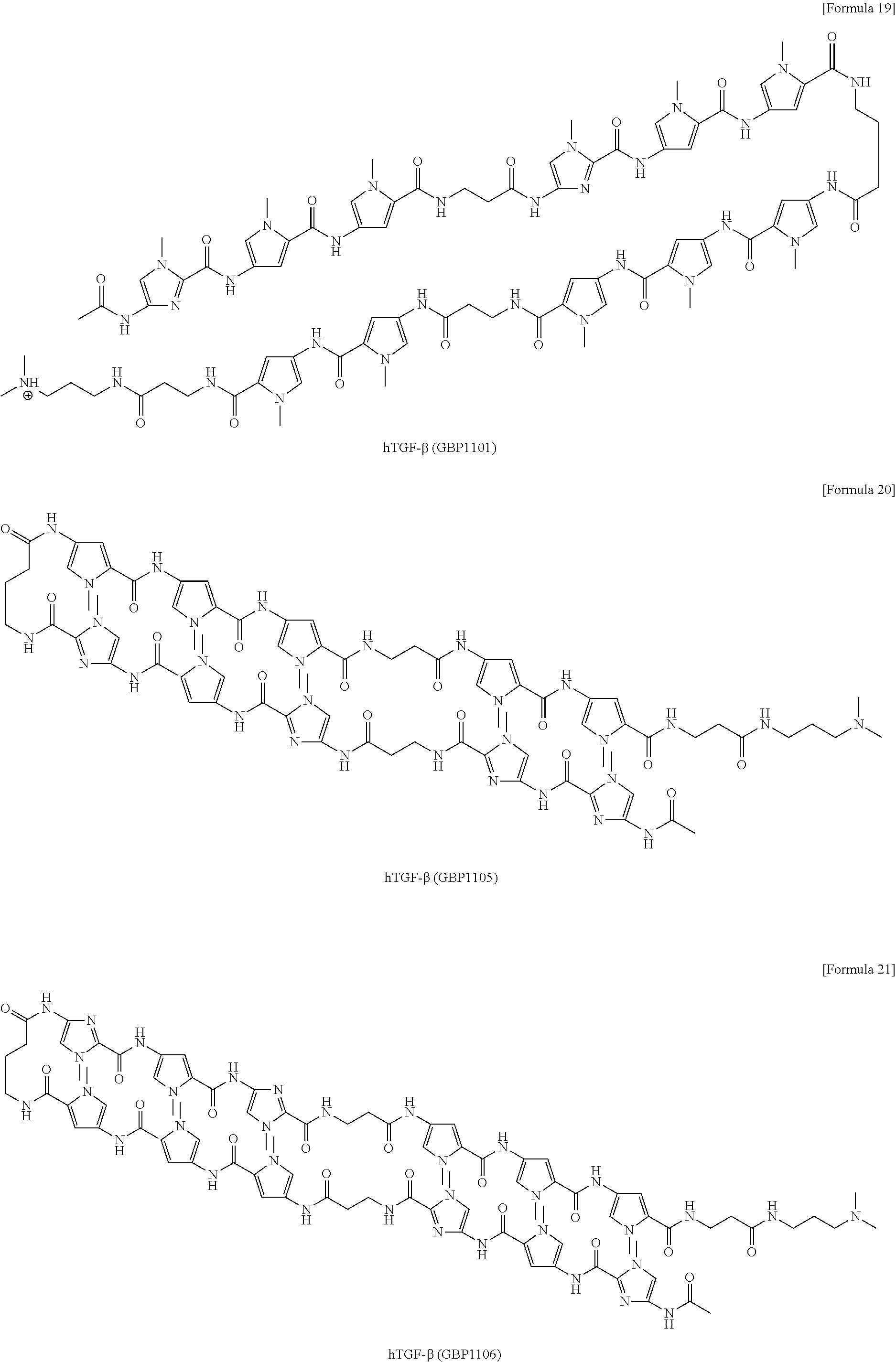

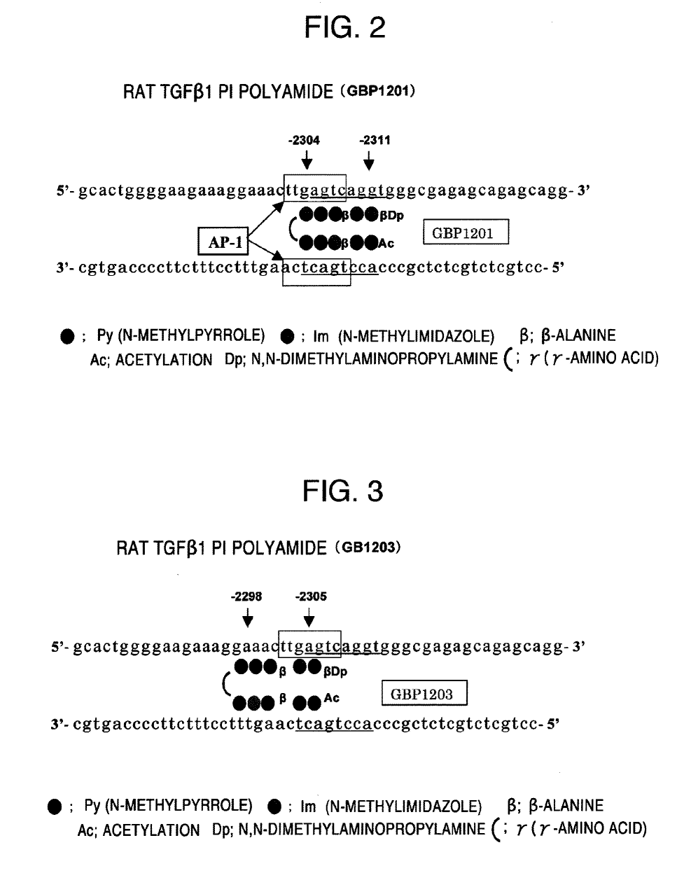

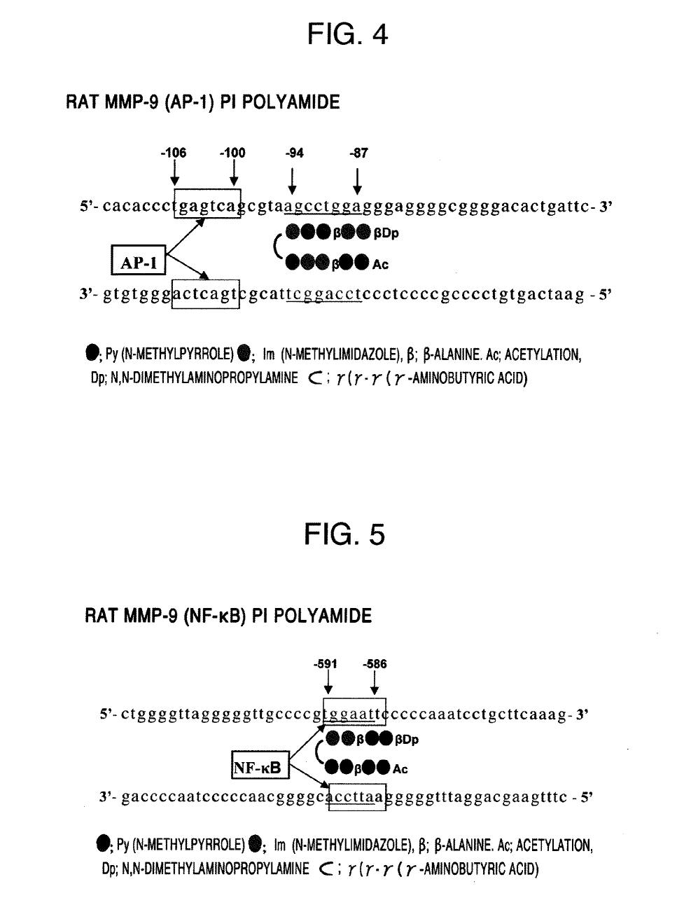

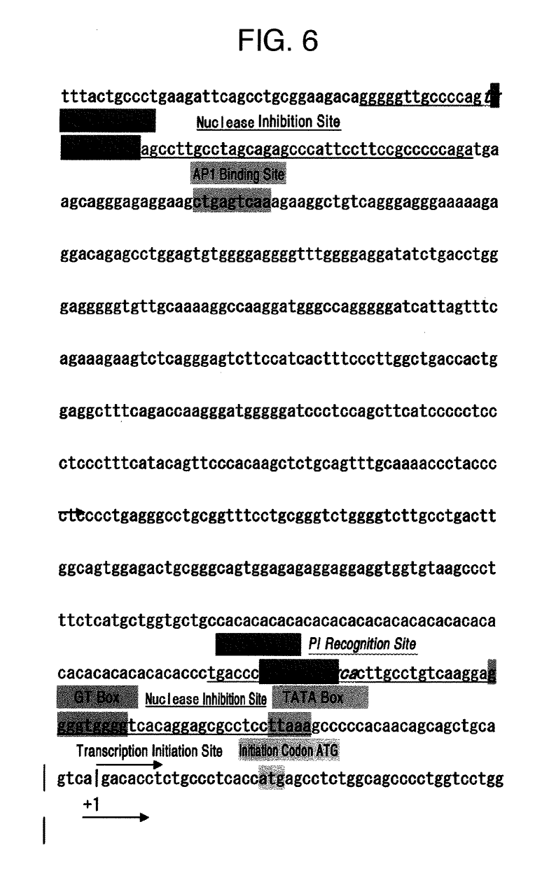

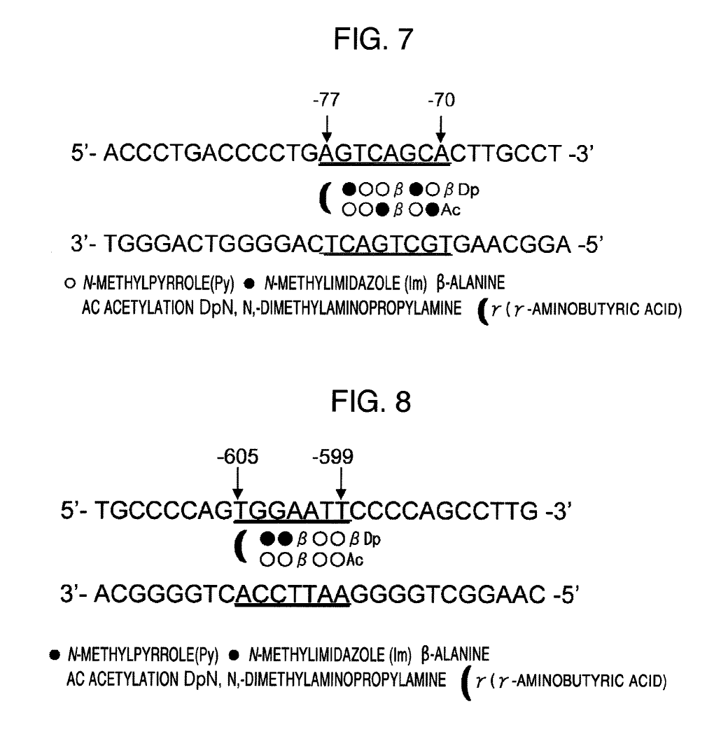

The nucleotide sequences of the gene regulatory regions of rat transforming growth factors β are shown in FIGS. 2 and 3.

The rat transforming growth factors β (GBP1201) and (GBP1203), the pyrrole-imidazole polyamide compounds of the present invention (hereinafter, also referred to as rTGF-β [GBP1201] and rTGF-β [GBP1203]) have a molecular formula of C₇₆H₉₃N₃₀O₁₅ and a molecular weight of 1666.7 and a molecular formula of C₇₈H₉₇N₂₈O₁₅ and a molecular weight of 1665.8, respectively. The target sequences of the rTGF-β gene regulatory regions (FIGS. 2 and 3) are a nucleotide sequence region from −2322 to −2293 (SEQ ID NO: 8) containing an AP1-binding sequence, more specifically a region from −2311 to −2304 (SEQ ID NO: 9), or a region from −2316 to −2287 (SEQ ID NO: 25), more specifically a region from −2305 to −2298 (SEQ ID NO: 26). Expression of the rTGF-β1 gene is inhibited by allowing the pyrrole-imidazole polyamide compounds to bind to the vicinity of the transcription factor binding site.

The pyrrole-imidazole polyamide rTGF-β (GBP1201) of the present invention is shown below.

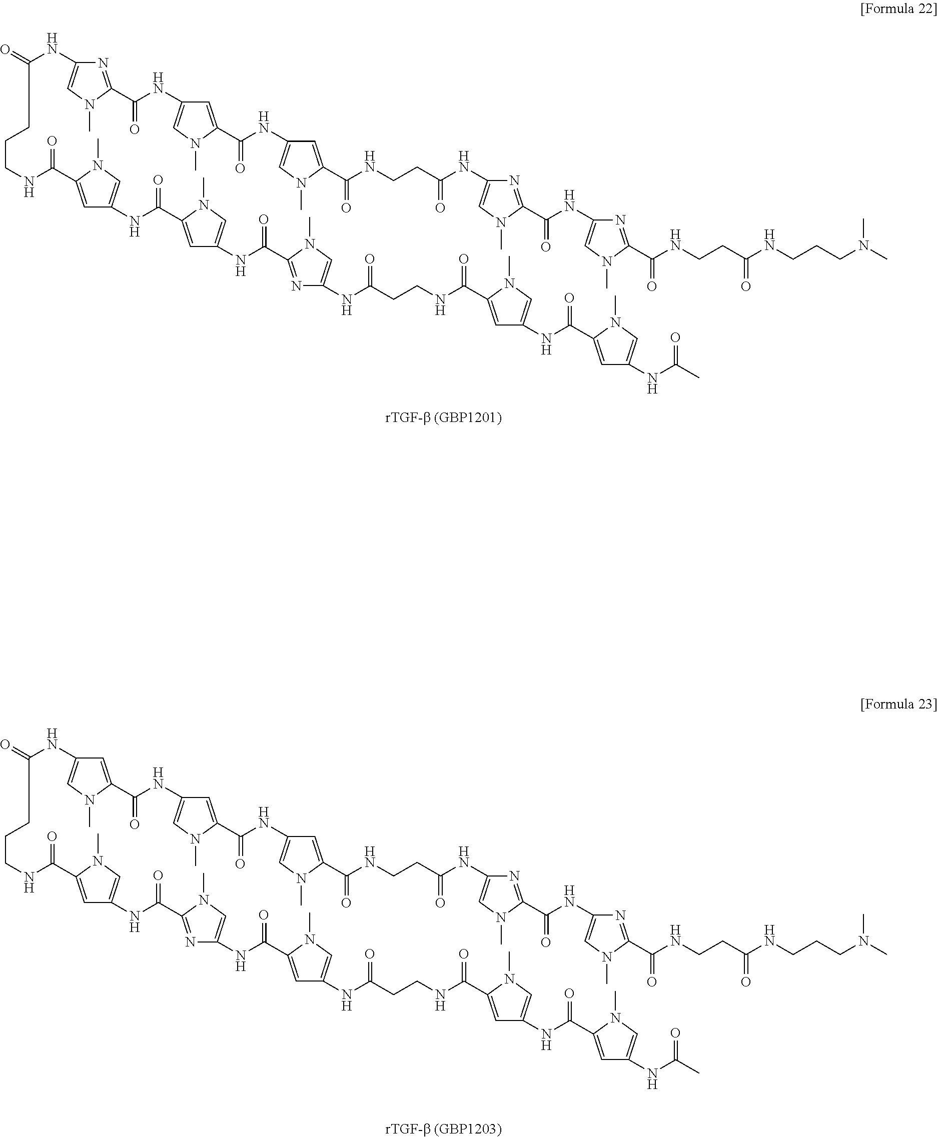

The human matrix metalloprotease 9AP1 (hereinafter, also referred to as hMMIP9AP1), the pyrrole-imidazole polyamide compound of the present invention, has a molecular formula of C₇₅H₉₃N₃₁O₁₅ and a molecular weight of 1668.6, and the target sequence thereof is a region from −88 to −59 (SEQ ID NO: 11) containing the AP1-binding region and the GT box regulatory region in a region from −653 to −24, the human matrix metalloprotease 9 gene regulatory region (SEQ ID NO: 10). More specifically, expression of human matrix metalloprotease 9 gene is inhibited by binding to agtcagca, 8 nucleotides in a region from −77 to −70 (SEQ ID NO: 12).

Human matrix metalloprotease 9 NFκβ (hereinafter, also referred to as hMMP9NFκβ), the pyrrole-imidazole polyamide compound of the present invention, has a molecular formula of C₆₆H₈₄N₂₄O₁₃, and a molecular weight of 1421.3, and the target sequence thereof is a region from −616 to −588 (SEQ ID NO: 13) containing the NFκβ region in a region from −653 to −24, the matrix metalloprotease 9 gene regulatory region (SEQ ID NO: 10). More specifically, expression of the matrix metalloprotease 9 gene is inhibited by allowing the pyrrole-imidazole polyamide compound to bind to tggaatt, 7 nucleotides in a region from −605 to −599 (SEQ ID NO: 14).

hMMP9AP1 and hMMPNFκβ, the pyrrole-imidazole polyamides of the present invention, are shown below.

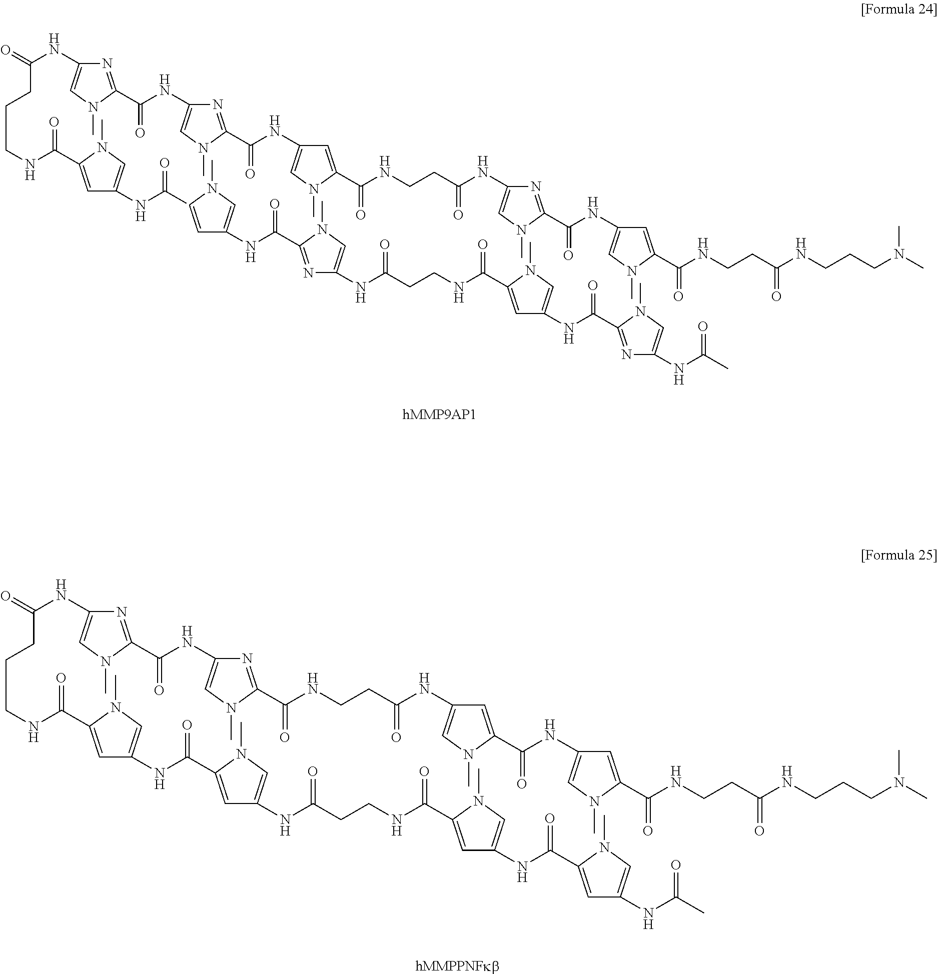

Rat matrix metalloprotease 9AP1 (hereinafter, also referred to as rMMP9AP1), the pyrrole-imidazole polyamide compound of the present invention, has a molecular formula of C₇₅H₉₃N₃₁O₁₅ and a molecular weight of 1668.74, and the target sequence thereof is a region from −105 to −76 (SEQ ID NO: 15) containing the AP1-binding region and the GT box regulatory region in the rat matrix metalloprotease 9 gene regulatory region. More specifically, expression of the matrix metalloprotease 9 gene is inhibited by allowing the pyrrole-imidazole polyamide compound to bind to 8 nucleotides from −94 to −87 (SEQ ID NO: 16).

Rat matrix metalloprotease 9NFκβ (hereinafter, also referred to as rMMP9NFκβ), the pyrrole-imidazole polyamide compound of the present invention, has a molecular formula of C₆₆H₈₄N₂₄O₁₃ and a molecular weight of 1421.5, and the target sequence thereof is a region from −602 to −575 (SEQ ID NO: 17) containing the NFκβ region in the rat matrix metalloprotease 9 gene regulatory region. More specifically, expression of the matrix metalloprotease 9 gene is inhibited by allowing the pyrrole-imidazole polyamide compound to bind to 6 nucleotides of a region from −591 to −586 (SEQ ID NO: 18).

rMMP9AP1 and rMMPNFκβ, the pyrrole-imidazole polyamides of the present invention, are shown below.

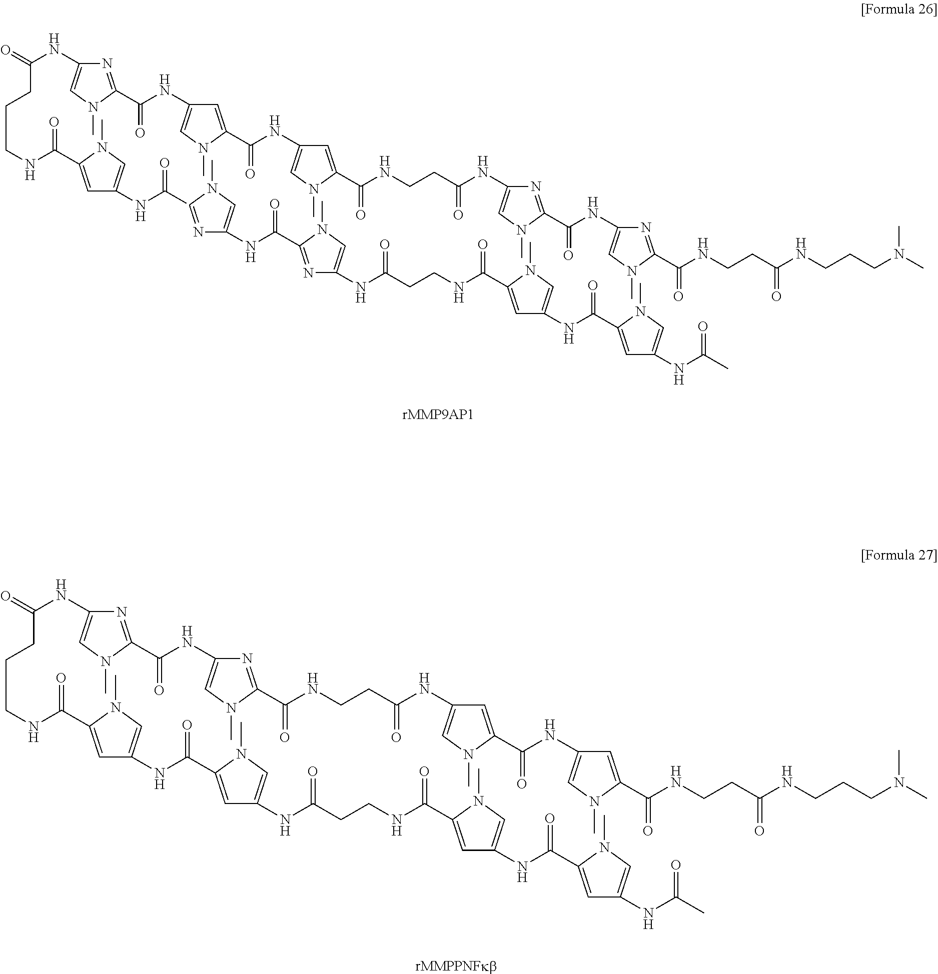

Since the matrix metalloprotease 9 gene sequence is highly conserved in mammal, the rMMP9AP1 compound and the rMMP9NFκβPIP compound can be used in humans.

In the present invention, proliferative ophthalmic diseases refer to cornea neovascularization associated with tumor, proliferative diabetic retinopathy, pterygium, neovascularization glaucoma, age-related macular degeneration, and cornea disease, but are not limited to these diseases.

In the present invention, inflammatory ophthalmic diseases refer to infiltration of corneal inflammatory cells associated with infections, keratitis, conjunctivitis, allergic diseases, and pollinosis, but are not limited to these diseases.

In the present invention, a topical therapeutic agent for an ophthalmic disease may be in any dosage form of therapeutic agents for an ophthalmic disease, such as a drug for intravitreal administration, a subconjunctival injection, an eye drop, and an ointment unless otherwise specified.

In the present invention, a topical therapeutic agent for an ophthalmic disease may contain pharmacologically acceptable general added ingredients, such as a diluent and an excipient.

The present inventors demonstrated that a trauma due to an alkali burn could be treated by administering to the eye a pyrrole-imidazole polyamide that could inhibit the transforming growth factor β gene and the matrix metalloprotease 9 gene selectively, not by intraocular injection.

Since a drug administered by instillation is usually washed away with a lachrymal fluid, and it is difficult to maintain an effective drug concentration in the cornea in conventional drug therapies for intraophthalmic diseases, frequent instillation is required. The present inventors demonstrated that the pyrrole-imidazole polyamide compound of the present invention administered by instillation was present in corneal cells continuously and healed the cloudiness in the eye after an alkali burn.

Furthermore, it was demonstrated in the measurement of the quantity of mRNA in the cornea by RT-PCR that the pyrrole-imidazole compound of the present invention passed through the cornea and inhibited expression of the TGF-β gene and the MMP-9 gene in the cornea.

It can be said that the data of the rat model shown in the present application also shows the same effect in the human eyes, another mammal.

Therefore, a topical therapeutic agent for an ophthalmic disease containing the pyrrole-imidazole polyamide compound of the present invention can be used for treatment without frequent instillation and has an advantage that risks for basement membrane digestion and fibroblast infiltration can be avoided because of the decreased effective concentration. Furthermore, there is also an advantage that the patient's QOL is improved significantly since treatment can be easily performed not by injection.

Furthermore, since the pyrrole-imidazole polyamide compound of the present invention is known to remain in the nucleus, the pyrrole-imidazole polyamide compound of the present invention does not need to be administered several times daily unlike a conventional instillation agent. For example, adequate effect is exhibited by once-daily instillation. Therefore, advantageous effects are exhibited.

Furthermore, since the pyrrole-imidazole polyamide compound of the present invention is a novel compound that complements defects of inhibitors of a target gene in the ophthalmologic field and selectively inhibits a gene in the eyeball and surrounding tissues, treatment for each disease that could not be solved by conventional therapies can be simply discovered by screening of a target compound.

Furthermore, as a therapeutic agent for an ophthalmic disease that requires treatment by a drug for vitreous administration or a local injection, the PIP compound of the present invention remains in the nucleus in a cell and does not need to be administered frequently as described above, and the hazard ratio of having an infection induced by frequent intraocular administration is decreased. Furthermore, adverse drug reactions by maintaining a high systemic drug concentration after intravenous administration can be prevented. Therefore, the pyrrole-imidazole polyamide compound of the present invention can be easily developed as an agent for vitreous administration or local injection.

Furthermore, the pyrrole-imidazole polyamide compound of the present invention can be used as a reagent for a function test in molecular biology research and drug development research in the ophthalmologic field because of the gene selecting effect thereof.

### EXAMPLES

### 1. Synthesis of Py-Im Polyamides Corresponding to Promoters

### (1) Designing of Py-Im Polyamides Corresponding to Transcription Regulatory Sites of Human and Rat Transforming Growth Factor β Genes and Human and Rat Matrix Metalloprotease 9 Genes

### I. Materials and Methods

The above-mentioned PIP compounds according to the present invention were designed as Py-Im polyamides.

### (2) Machine-Assisted Automated Synthesis of Py-Im Polyamide Using the Fmoc Method

Machine-assisted automated synthesis of a pyrrole-imidazole polyamide was performed using a sequential flow peptide synthesizer Pioneer (trade name) (Applied Biosystems) with 0.1 mmol scale (200 mg of Fmoc-β-alanine-CLEAR acid resin, 0.50 meq/g, Peptide Institute, Inc.). The automated solid phase synthesis consists of DMF wash, removal of a Fmoc group with 20% piperidine/DMF, methanol wash, coupling with a monomer for 60 minutes in the presence of HATU and DIEA (4 equivalents each), methanol wash, if necessary, protection with acetic anhydride/pyridine, and final DMF wash. The Py-Im polyamide was generally obtained with a moderate yield (10% to 30%).

FITC coupling: 4 times excess fluorescein (0.40 mmol) and DIEA (no HATU) dissolved in DMF was flashed through a column over 60 minutes.

Common procedure: An Fmoc group was removed from an Fmoc-β-alanine-Wang resin, and then the resin was continuously washed with methanol. The coupling step was performed with an Fmoc amino acid. Subsequently, the resin was washed with methanol. All these steps were repeated many times until all the sequences were introduced. After the coupling step was completed, if necessary, the N terminal amino group was protected or coupled with FITC, and washed with DMF, and the reaction container was removed.

Degradation as carboxylic acid: A synthetic polyamide was isolated with a cool ethyl ether precipitate after the degradation step (5 mL of a mixture of 91% TFA-3% TIS-3% DMS-3% water/0.1 mmol of resin).

Degradation as amine: A synthetic polyamide was isolated with a cool ethyl ether precipitate after the degradation step (5 mL of N,N-dimethylaminopropylamine/0.1 mmol of a resin, 50° C., overnight).

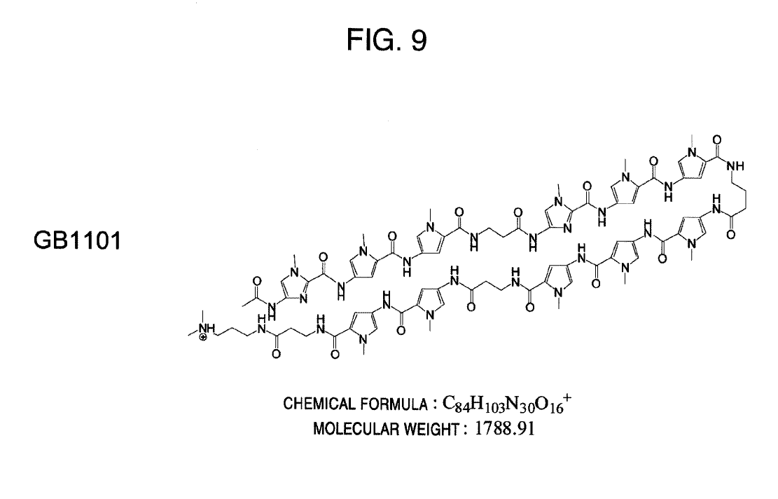

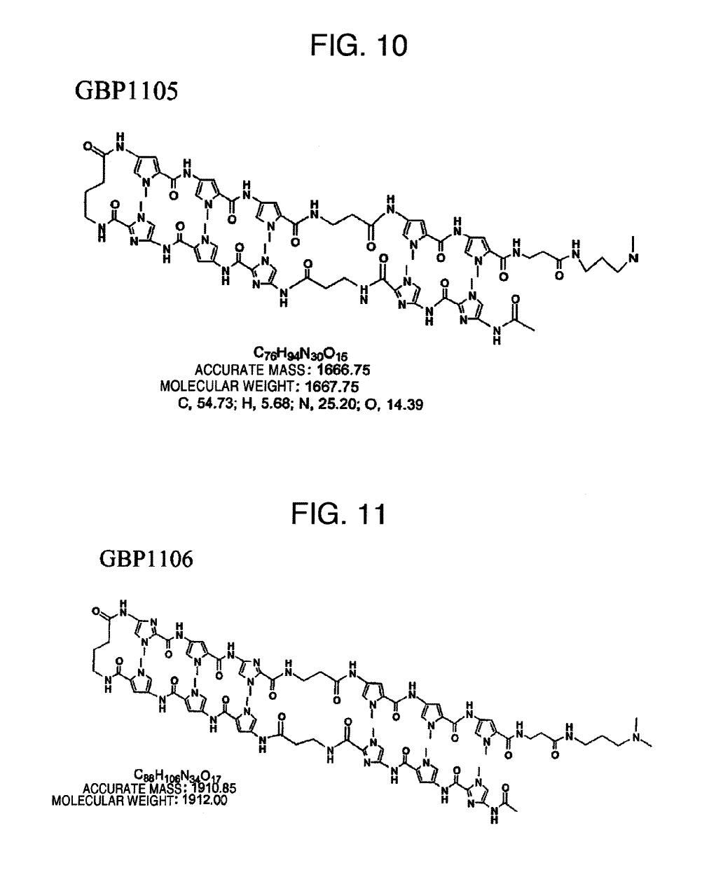

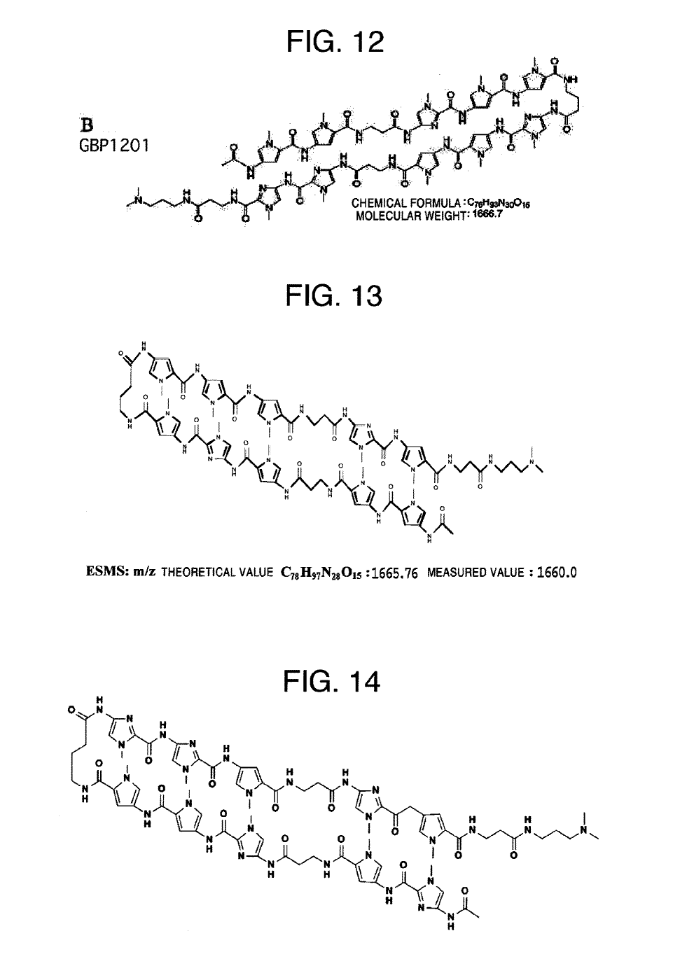

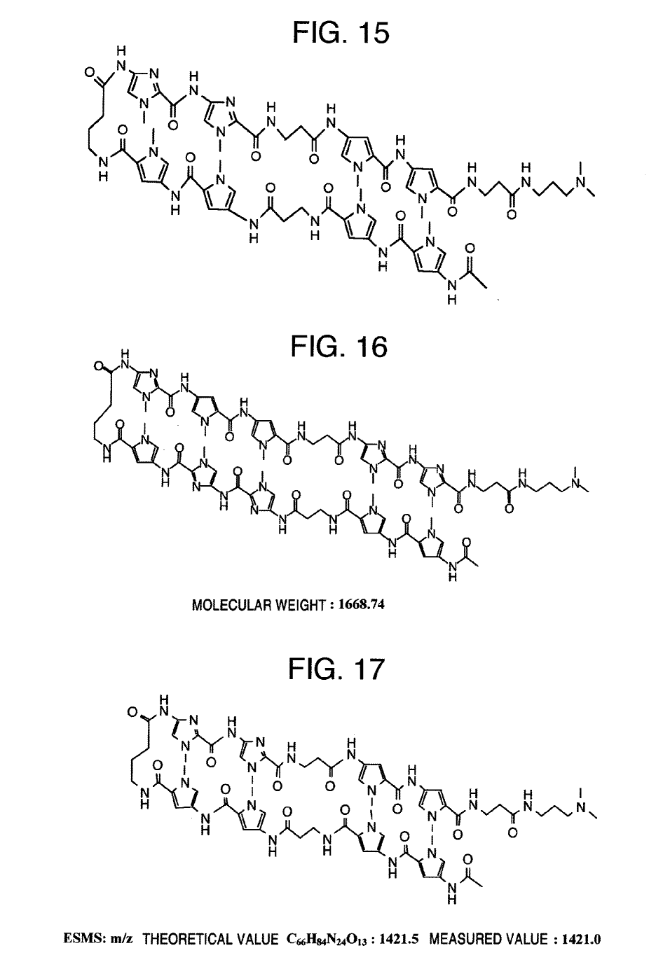

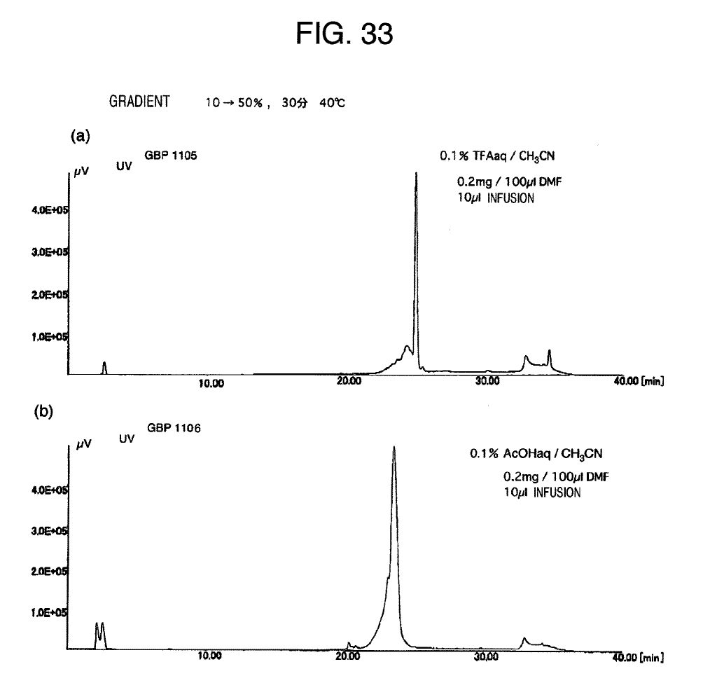

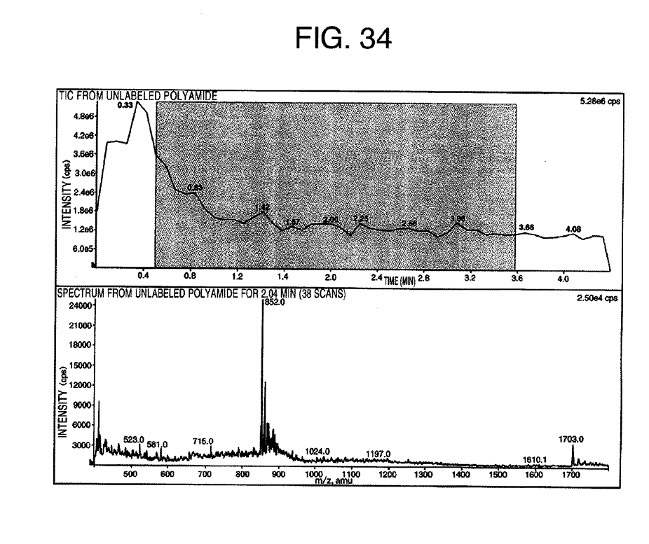

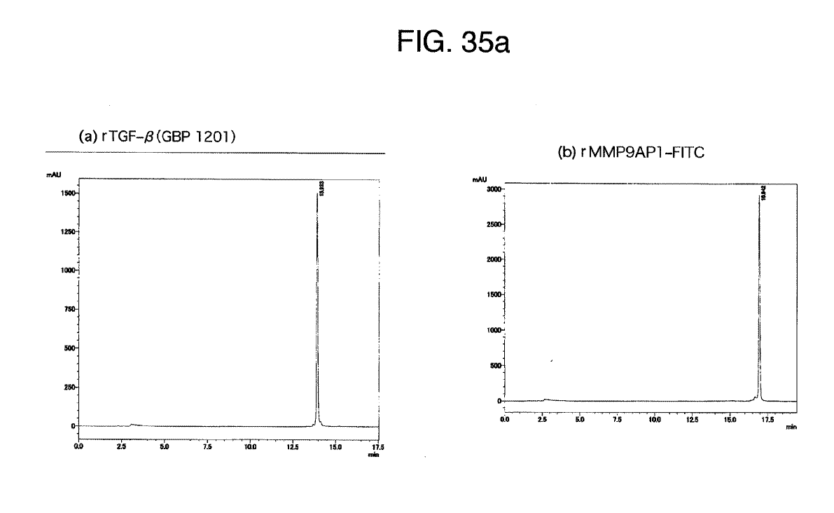

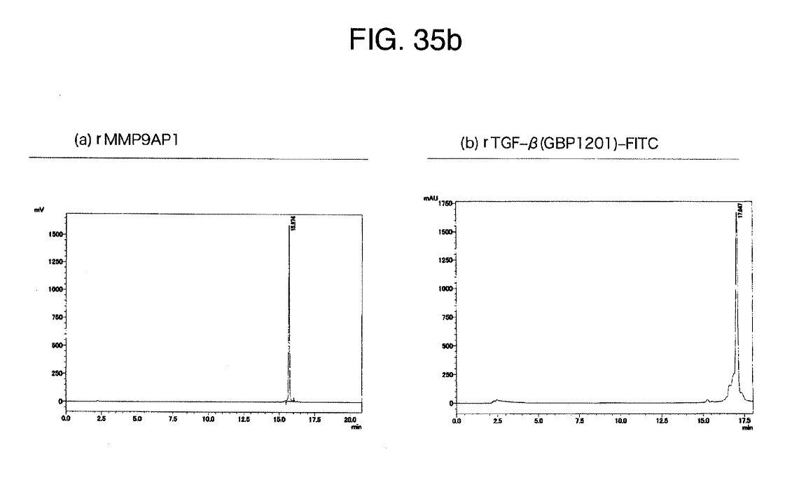

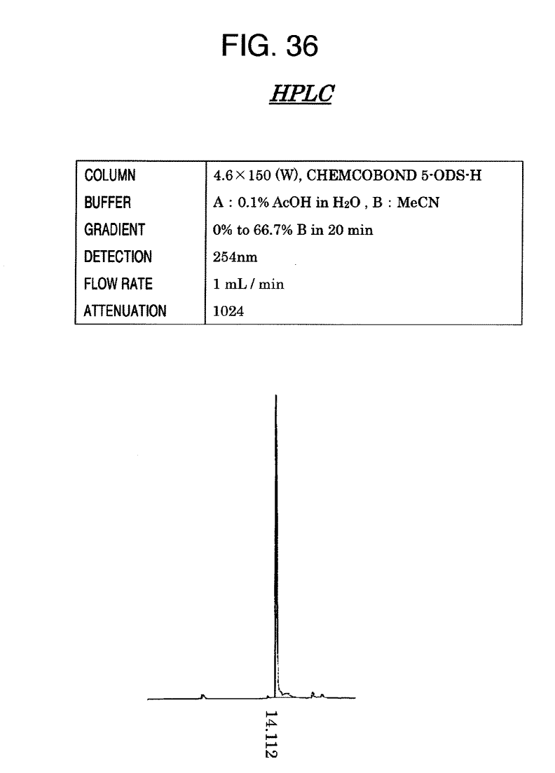

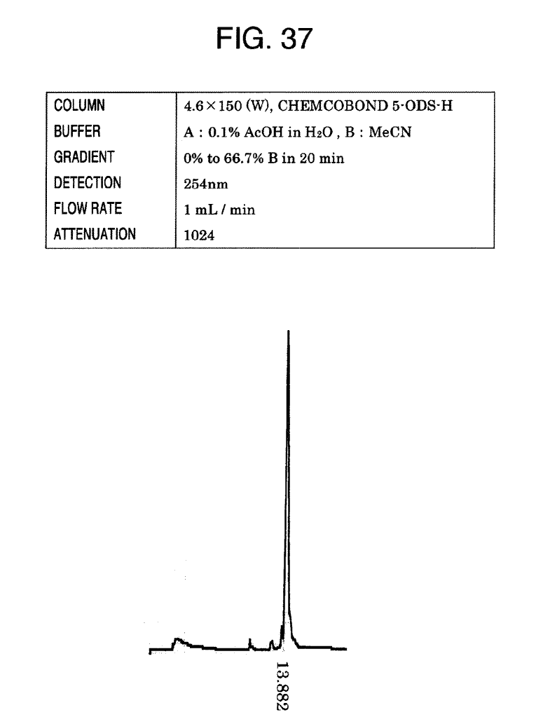

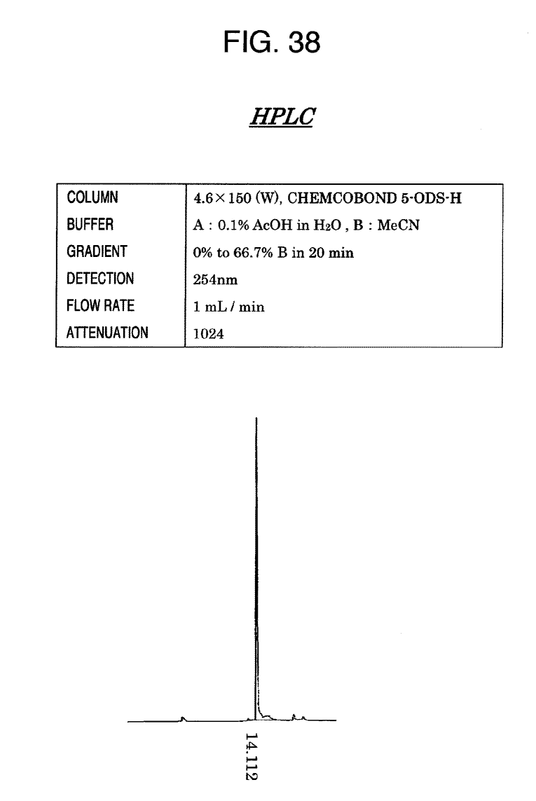

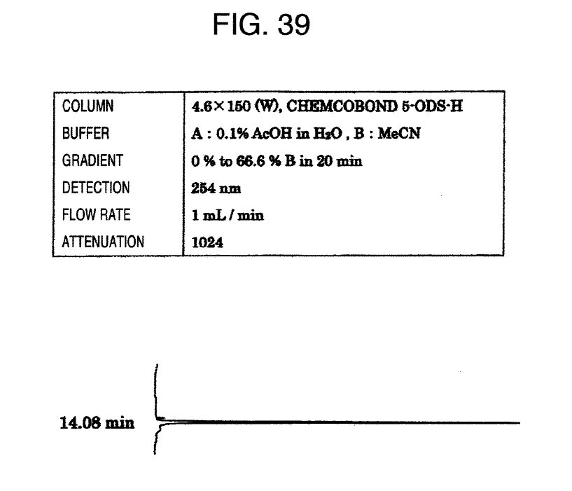

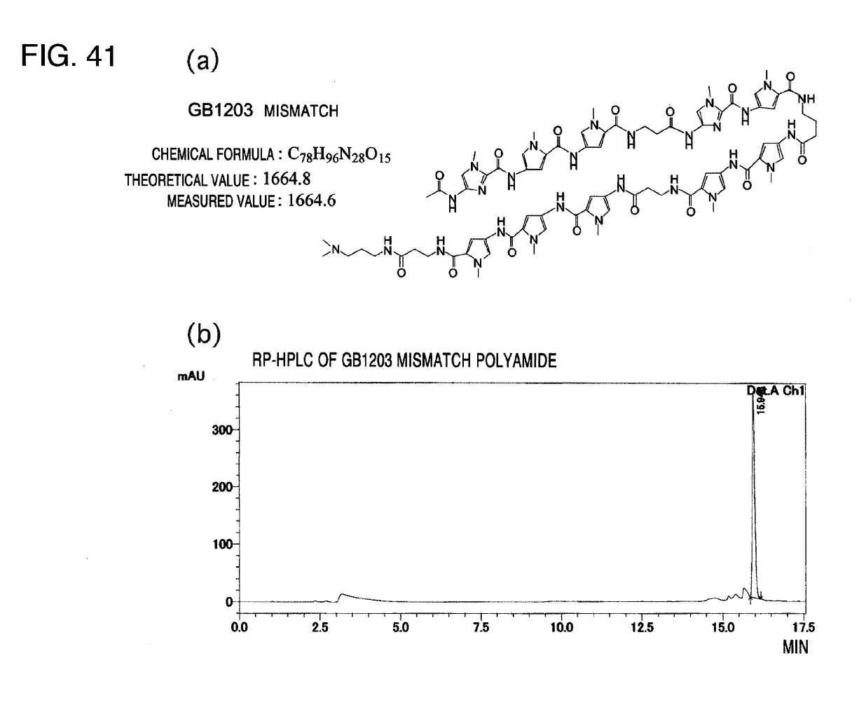

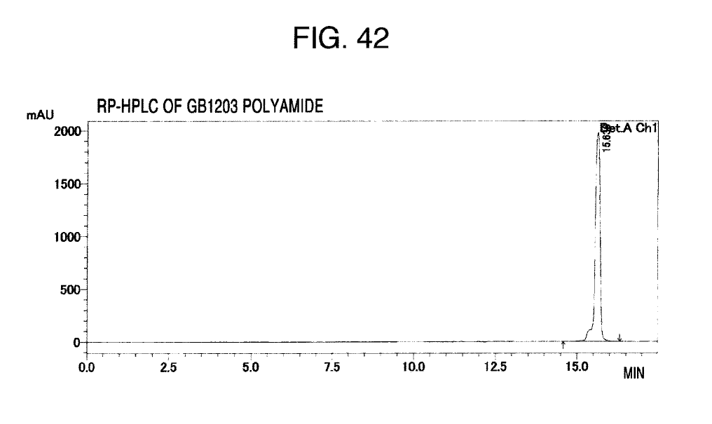

Purification: The final purification was performed by UV detection at 350 nm using a linear gradient of B (acetonitrile) in buffer A (0.1% TFA/water or 0.1% AcOH/water) by RP-HPLC for analysis at a flow rate of 10 mL/min and UV detection at 254 nm using a linear gradient of B (MeCN) in buffer A (0.1% AcOH/water) by RP-HPLC for analysis at a flow rate of 1 mL/min. hTGF-β (GBP1101), hTGF-β (GBP1105), hTGF-β (GBP1106), rTGF-β (GBP1201), rTGF-β (GBP1203), hMMP9AP1, hMMP9NFκβ, rMMP9AP1, rMMP9NFκβ, rTGF-β (GBP1201)-FITC, rTGF-β (GBP 1201) mismatch, rMMP9AP1-FITC, and rTGF-β (GBP1203) mismatch are shown in FIGS. 9, 10, 11, 12, 13, 14, 15, 16, 17, 27, 28, 29, and 41a. The RP-HPLC charts of hTGF-β (GBP1105) and hTGF-β (GBP1106) are shown in FIG. 33. The total ion chromatogram (TIC) chart and electrospray ionization mass spectrometry spectrum of hTGF-β (GBP1101) are shown in FIG. 34. The RP-HPLC charts of rTGF-β (GBP1201) and rMMP9AP1-FITC are shown in FIG. 35a. The RP-HPLC charts of rMMP9AP1 and rTGF-β (GBP1201) FITC are shown in FIG. 35b. The RP-HPLC chart of rMMPNFκβ is shown in FIG. 36. The RP-HPLC chart of hMMP9AP1 is shown in FIG. 37. The RP-HPLC chart of hMMP9NFκβ is shown in FIG. 38. The RT-PCR chart of rTGF-β (GBP1201) is shown in FIG. 39. The RP-HPLC chart of rTGF-β (GBP1203) mismatch is shown in FIG. 41(b). The RT-PCR chart of rTGF-β (GBP1203) is shown in FIG. 42.

### 2. Preparation of Alkali Burn Animal Model

8-week-old male Wister rats were prepared and anesthetized with diethyl ether. Then, 5 μl of 0.5 N sodium hydroxide solution was administered for anesthesia to adult Wister rats by instilling the central part of the cornea of both eyes or one eye by a common method according to Saika et al. (Am J. Patho., 2005). At 10 seconds after instillation, the alkali liquid was washed off from the cornea surface with 10 ml of phosphate buffer to prepare an alkali burn rat model.

3. Binding Specificity and Affinity of rTGF-β1 (GBP1201) PIP for Target DNA

### I. Method

For the gel shift assay, a fluorescence-labeled match oligo DNA containing the AP-1 binding site that corresponds to the TGF-β1 promoter was synthesized. Sense and antisense oligo DNA suitable for the test were annealed to prepare a double-stranded oligo DNA. 1 μM DNA was incubated with 50 μM PIP compound at 37° C. for 1 hour, separated by electrophoresis using 20% polyacrylamide gel, and visualized using a fluorescence image analyzer LAS-3000 (Fuji Film, Tokyo, Japan).

Biacore assay can measure binding affinity of rTGF-β (GBP1201) PIP for a target DNA in real time.

Biotin-labeled oligo DNA were annealed to form a double strand, and the double strand was immobilized on a sensor chip SA (Biacore, Uppsala, Sweden) on which streptavidin was immobilized beforehand.

Dynamics of interactions of rTGF-β (GBP1201) PIP, mismatch (FIG. 41 [a]) and biotin-labeled oligo DNA were investigated using Biacore 2000 System (Biacore, Uppsala, Sweden). Data was processed according to the protocol recommended by Biacore2000.

### II. Results

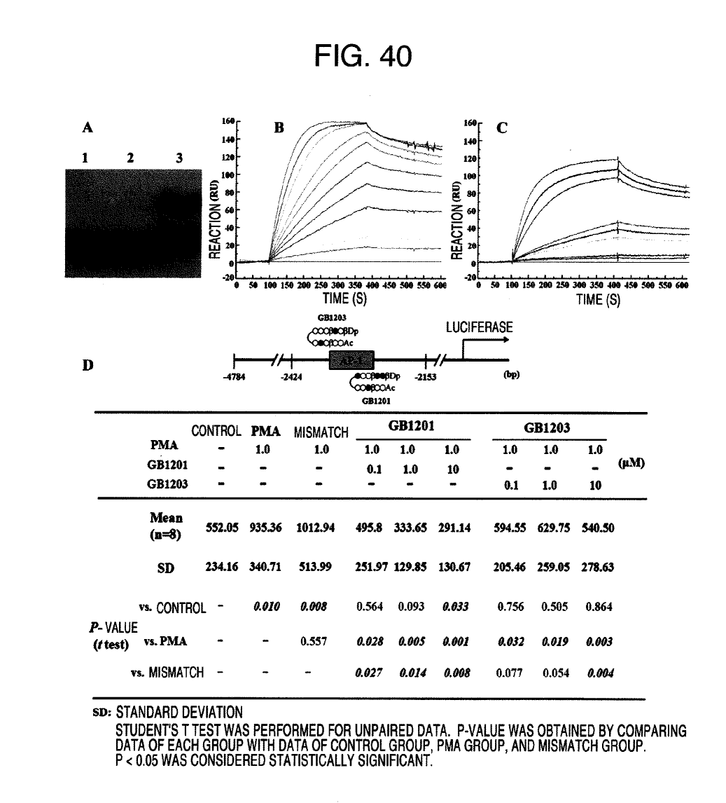

The gel shift assay showed that the rTGF-β (GBP1201) PIP of the present invention had bound specifically to the target DNA (FIG. 40). On the other hand, the rTGF-β (GBP1201) PIP of the present invention did not bind to the mismatch polyamide.

By the Biacore assay, dynamic analysis was performed for rTGF-β (GBP1201) PIP and mismatch polyamide. From the surface plasmon resonance sensorgram, rTGF-β (GBP1201) PIP showed a high binding affinity for target DNA, 149 times the affinity for the mismatch polyamide, and KD=5.12±3.43 E-09 was calculated.

4. Effect of the Pyrrole-Imidazole Polyamide of the Present Invention in Rats with Alkali Burn

### I. Method

At 1 hour after a trauma due an alkali burn was induced (9.00 am), and 1 μmol/L rTGF-β (GBP1201) PIP compound and 1 μmol/L of a mixture of rMMP9AP1 and rMMP9NFkB in 1:1 (hereinafter, also referred to as rMMP9PIP) dissolved in 5 μl of 0.1% acetic acid hydrate were administered to the rat right eye by dropping in the central part of the cornea at 9.00 am once daily every day for 1 week. As a control, 0.1% aqueous acetic acid solution was administered to the left eye in the same manner as described above. The same experiment was performed in 6 rats. The reason why 0.1% aqueous acetic acid solution was used was that the PIP compound of the present invention was dissolved in this aqueous solution. Furthermore, to prevent the occurrence of an infection, antibiotic eye ointment Tarivid Ophthalmic Ointment 0.3% (Santen Pharmaceutical Co., Ltd.) was administered at 5.00 pm every day. Furthermore, the drug was administered to 2 rats in which an alkali burn was not induced as controls. Rats were observed every day at the time of instillation and application of an eye ointment, and the cornea was observed and photographed every evening. Water-soluble fluorescein has a property of staining only such an ulcer or a wound lesion in which the stromal layer is exposed. By utilizing this property, the corneal injury was evaluated according to the cornea fluorescein test using a FLUORES test paper (Showa Yakuhin Kako Co., Ltd.) that can test the corneal condition.

Transparency of the cornea was observed with a microscope every day. The cornea was observed until complete reepithelialization was observed. When the fluorescence staining of the cornea was not observed, complete reepithelialization was defined.

### Evaluation of Opacity and Ulceration of the Cornea

Transparency of the cornea was evaluated as follows.

Grade 0: No opacity.
Grade 1: Less than one third of the cornea surface is clouded.
Grade 2: Less than two thirds of the cornea surface is clouded.
Grade 3: Two thirds or more of the cornea surface is clouded.
Grade 4: Most of the surface is clouded, and visualization of the margin of the pupil margin is prevented by cloudiness.

### Evaluation of Severity of Corneal Defect

Grade 0: No defect or ulceration
Grade 1: Ulceration is limited to one third of the anterior part of the cornea. Grade 2: Ulceration is extended to one third of the central part of the cornea.
Grade 3: Ulceration is extended to one third of the posterior part of the cornea.
Grade 4: Formation of descemetocele

### Grade 5: Perforation

### II. Results

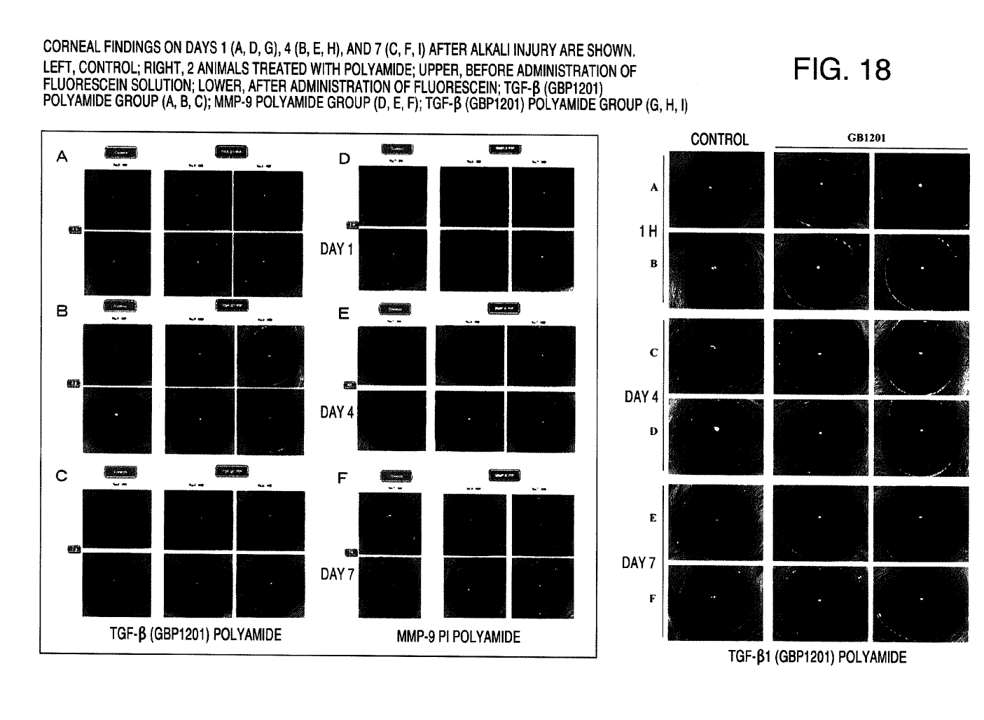

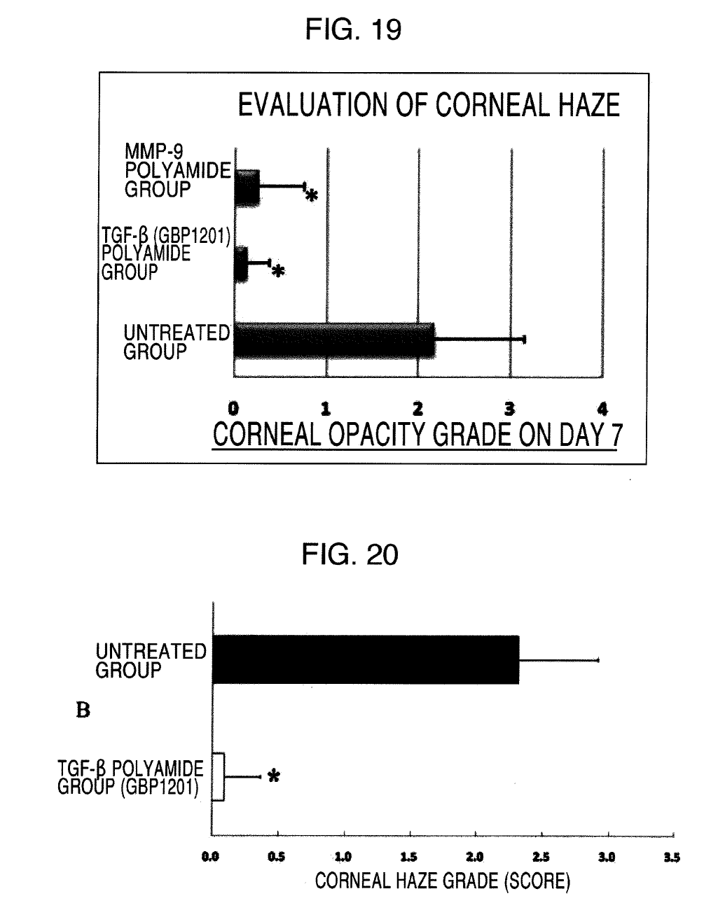

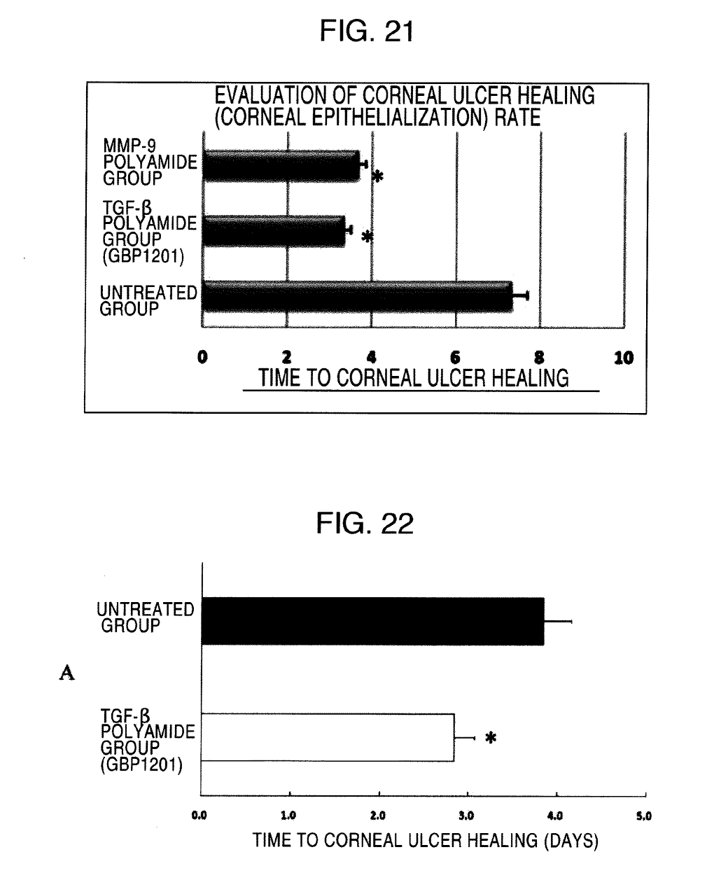

Corneal findings after an alkali injury in rats are shown in FIG. 18. The results of evaluation of corneal opacity and ulceration are shown in FIGS. 19 and 21. The results of evaluation of severity of the corneal defects are shown in FIGS. 20 and 22. The rTGF-β (GBP1201) PIP compound and rMMP9PIP healed corneal opacity, ulceration, and corneal defect significantly better than the control.

5. Real Time RT-PCR Assay (Measurement of mRNA Expression)

### I. Material and Method

4 rats were treated with alkali, then rTGF-β (GBP1201) polyamide and rMMP9PIP were dissolved in 0.1% acetic acid solution, the concentration was adjusted to 1 μMol/L, and 5 μl was dropped in the central part of the cornea at 1 hour after trauma induction. Animals were slaughtered 24 hours, 5, 10, and 30 days later. For the groups treated with the rTGF-β (GBP1201) PIP compound or the rMMP9PIP compound and the control group, a 3-mm disposable biopsy punch was used to excise the injured cornea region. According to the method described in Saika. et al., Am J Patho, 2005 and Saika S. et al., Laboratory Investigation, 2005, total RNA was prepared, quantitative RT-PCR was performed, and TGF-β1 and MMP-9 mRNA levels were evaluated.

The complementary DNA chain prepared by reverse transcription was synthesized with a SuperScript™ First-Strand Kit (Invitrogen Life Technologies, Corp.). SYBR Premix Ex Taq Kit (Takara Bio Inc.) was used for real time RT-PCR (Thermal Cycler Dice Real Time System TP800, Takara Bio Inc., Japan). As primers for TGF-β1, a forward primer of 5′-CCA AGG AGA CGG AAT ACA GG-3′ (SEQ ID NO: 19) and a reverse primer of 5′-AGC TGT GCA GGT GTT GAG C-3′ (SEQ ID NO: 20) (194-bp PCR products) were used. As primers for MMP-9, a forward primer of 5′-TTC GAC GCT GAC AAG AAG TG-3′ (SEQ ID NO: 21) and a reverse primer of 5′-AGG GGA GTC CTC GTG GTA GT-3′ (SEQ ID NO: 22) (156-bp PCR products) were used. As primers for GAPDH, a forward primer of 5′-ACA TCA AAT GGG GTG ATG CT-3′ (SEQ ID NO: 23) and a reverse primer of 5′-GTG GTT CAC ACC CAT CAC AA-3′ (SEQ ID NO: 24) (161-bp PCR products) were used. 25 μl of two-step RT-PCR mixture consisted of 12.5 μl of SYBR Premix Ex Taq, 0.5 μl each of a forward and a reverse primer, 10.5 μl of RNase free water, and 1 μl of template cDNA. The real time cycle conditions were a reaction system of 95° C. for 10 seconds, 95° C. for 5 seconds, and at 60° C. for 30 seconds. Quantification of the TGF-β1 gene and the MMP-9 gene was standardized by comparing with GAPDH expression.

### II. Results

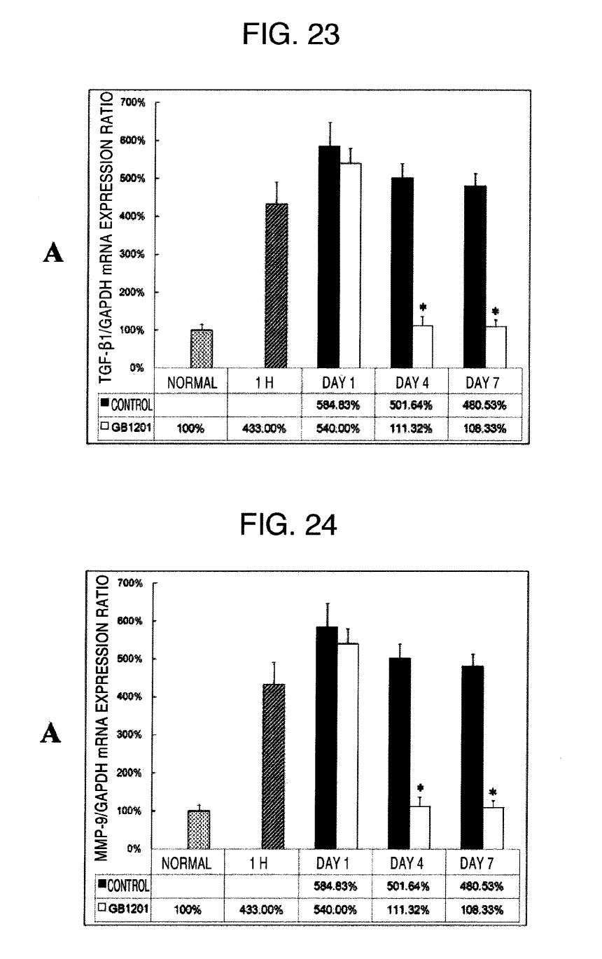

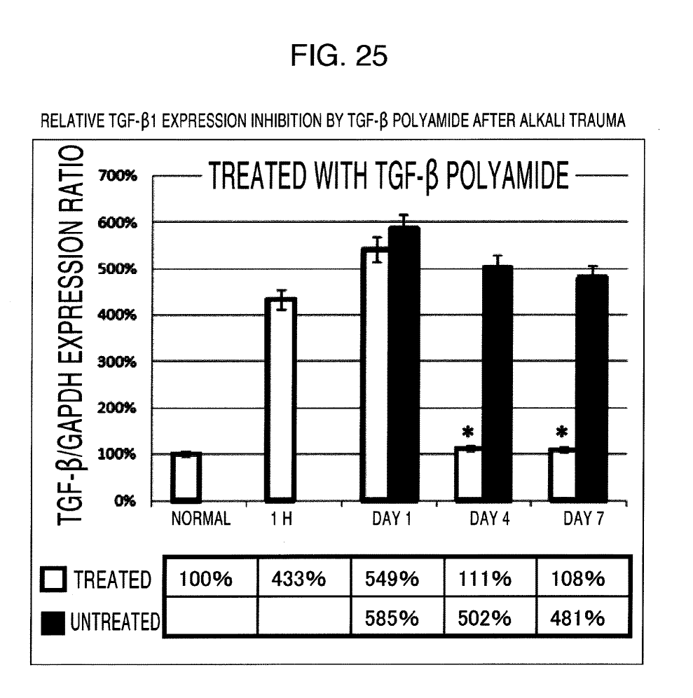

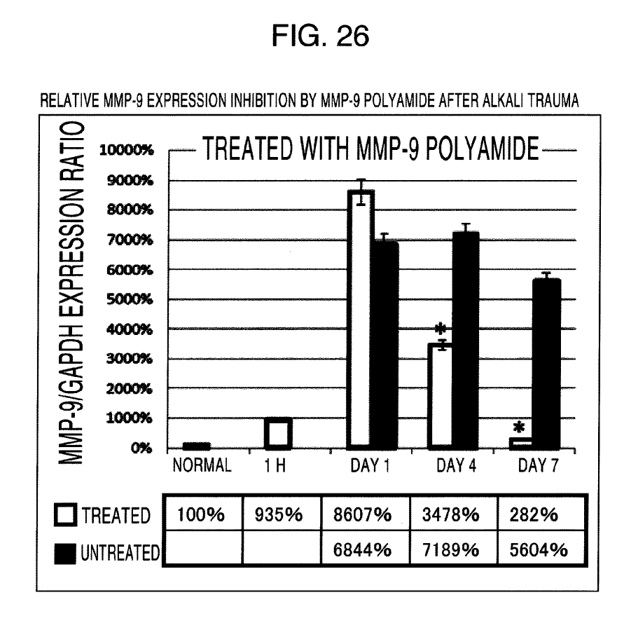

To investigate whether the polyamide of the present invention correlated with decreased expression of TGF-β and MMP-9 mRNA in the rat cornea, TGF-β1 and MMP-9 mRNA expression levels in rat cornea cells treated with the polyamide of the present invention and controls treated with 0.1% aqueous acetic acid solution were compared by real time RT-PCR. The analysis by real time RT-PCR showed that TGF-β mRNA and MMP9 mRNA in the rat cornea were significantly decreased by treatment with the TGF-β PIP compound and the MMP9PIP compound (FIGS. 23 to 26).

6. Effect of Rapid Delivery of the Pyrrole-Imidazole Polyamide of the Present Invention into the Nucleus in Cells in the Cornea in Rats with Alkali Burn

### I. Method

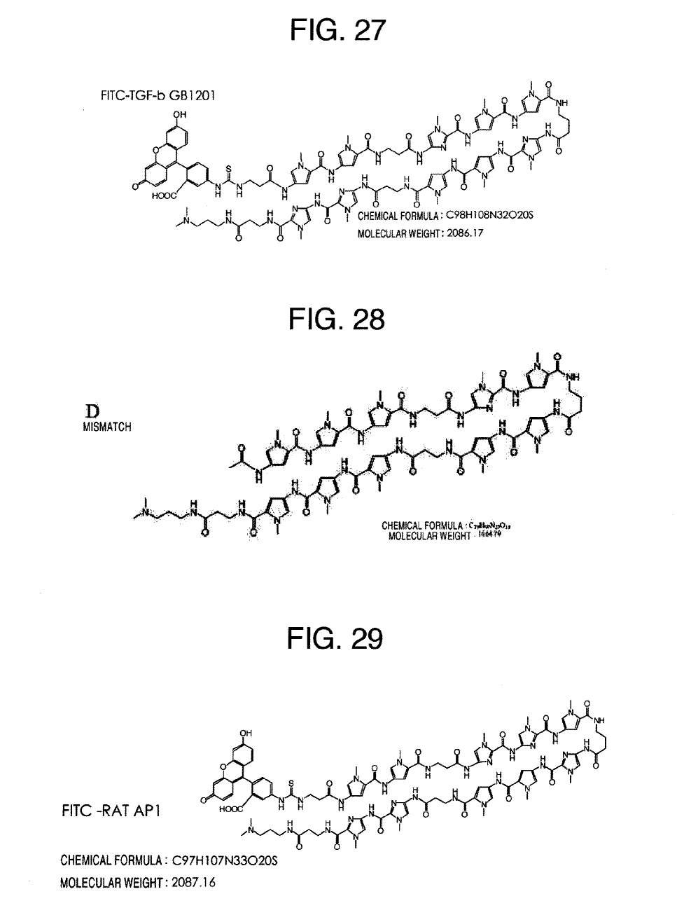

To examine penetration of the rTGF-β (GBP1201) polyamide, the rMMP-9NFkB polyamide, and the rMMP9AP1 polyamide into the nucleus of cells in the cornea, an FITC polyamide was synthesized by labeling each polyamide with Fluorescein isothiocyanate isomer-I (FITC) (FIGS. 27 and 29). The synthesized FITC polyamide was dissolved in 0.1% acetic acid solution, the concentration was adjusted to 1 μmol/L, 5 μl of the solution was dropped on the central part of the cornea of 8 alkali injury model rats at 1 hour after induction of a trauma. Two animals each were euthanized at 1 hour and on 1, 4, and 7 days after instillation of the FITC polyamide. The eyeball was isolated, embedded in the OCT compound (Miles, Elkhart, Ind., USA) in a plastic container for a frozen section sample, frozen with liquid nitrogen, and stored in an ultra-low temperature refrigerator at −80° C. The sample was thinly sliced into a section of 5 μm and observed under a fluorescence microscope (Olympus Corporation).

### II. Results

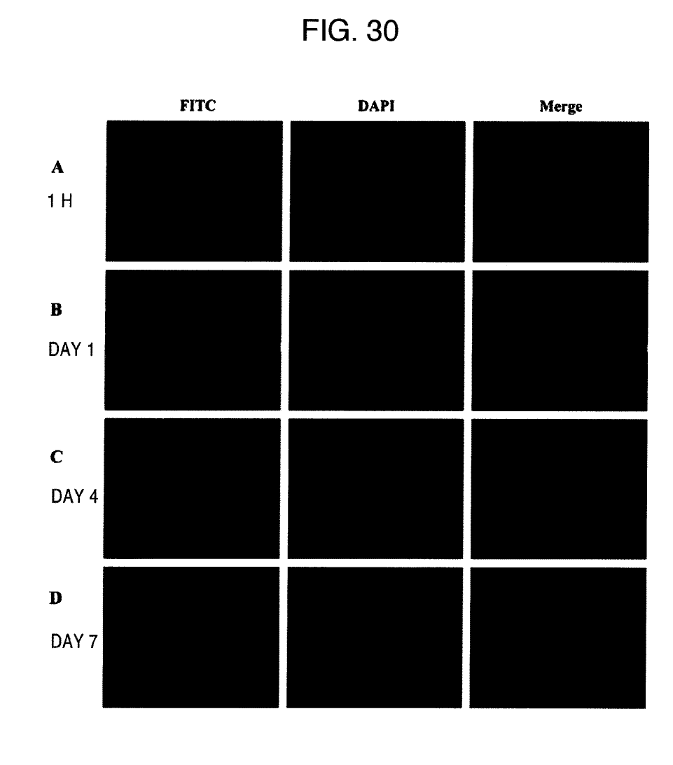

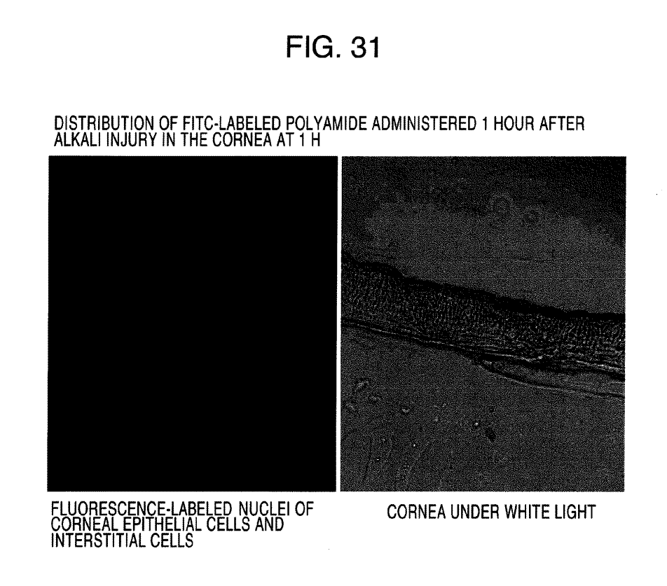

The polyamide of the present invention appeared to penetrate into the cornea rapidly and reach the inside of the nucleus of virtually all the cells in the cornea. Furthermore, after a single dose administration, fluorescence could be observed in the nucleus in some cells after 1 week. FIGS. 30A and 31 show cases where green FITC fluorescence is observed in the corneal epithelium and in the nucleus of interstitial cells in the cornea during the fluorescence observation at 1 hour after administration of the FITC-labeled rTGF-β (GBP1201) polyamide. The white part on the left of the figure is the nucleus of cornea cells, which looks white because of fluorescence FITC. The left part of the figure is the same corneal specimen with a white light. Intense fluorescence from FITC was observed at the same position as DAPI staining in all the cornea tissues, corneal epithelium cells, membrane keratocytes, and epithelial cells. This suggests that, in the use of the present invention, the polyamide compound would show drug efficacy continuously without being washed with a lachrymal fluid drained although the number of doses is reduced. In fact, this was also demonstrated because once-daily administration showed an adequate effect in the rat alkali injury models. From these results, rTGF-β (GBP1201) penetrated from the injured membrane epithelium layer into the corneal interstitial layer and epithelial layer and was distributed to the nucleus of all cells. The fluorescence signal was also detected in the anterior chamber. Furthermore, rTGF-β (GBP1201) remained in the nucleus even after 24 hours at a detectable level in cornea cells (FIG. 30B).

### 7. Immunohistochemistry Test

### I. Method

At 1 hour and on 1, 4, and 7 days after induction of a corneal injury in rats, the eyeball was isolated and embedded in the OCT compound (Miles, Elkhart, Ind., USA.). The frozen section was sliced into sections having a thickness of 5 μm, and histological and immunohistological analyses were performed. Using rabbit-anti-human Tgf-β1 polyclonal antibody (Yanaihira, Japan, catalog No. Y241) as a primary antibody, the sample was diluted 1000-fold before use. 4 nontrauma corneas as normal controls were used to compare rTGF-β (GBP1201) PIP-treated eyes and mock-treated eyes. To determine the specificity of the primary antibody, a negative control was also included.

### II. Results

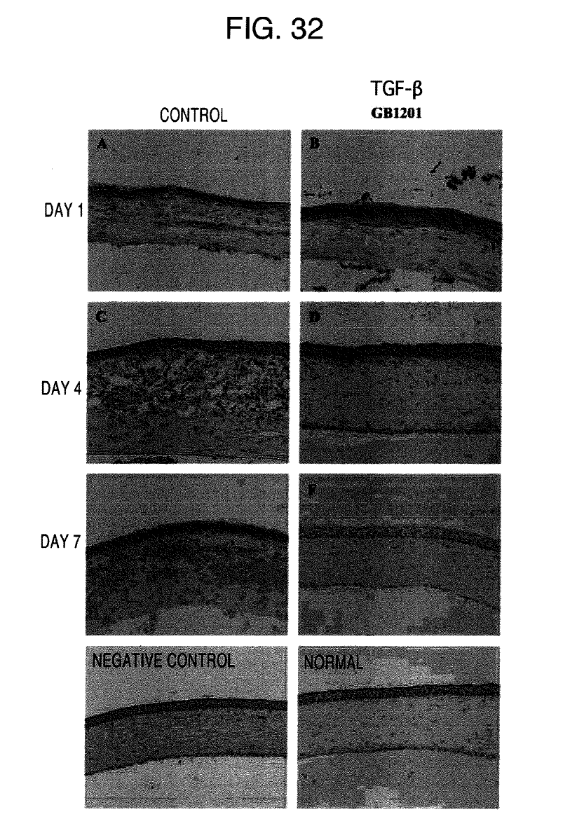

The immunohistochemical analysis showed that the TGF-β1 protein was expressed in the cornea free of a trauma although only a small amount is expressed, and the expression was detected only in the epithelium layer (FIG. 32, normal). After an alkali injury, the corneal epithelium layer was destroyed and completely lost. The corneal keratocytes were activated and proliferated to repair such a defect. On day 1, TGF-β1 was expressed in the activated anterior corneal stroma strongly. No immunohistological difference was noted between the rTGF-β (GBP1201) PIP and the controls (FIGS. 32A and 32B). On day 4, a strong TGF-β1 immune reaction was detected in both epithelial cells with a regenerated corneal stroma layer and proliferated corneal keratocytes (FIG. 32C). In the rTGF-β (GBP1201)-treated cornea (FIG. 32D), a weak TGF-β1 immune reaction was detected in epithelial cells with a regenerated corneal stroma layer. Less cell-mediated layered epithelia were noted in the corneal stroma. It was found that infiltration of inflammatory cells had not occurred, and fibrous cells had not increased locally. In the control group, the corneal stroma was expanded thickly, and spindle-shaped Tgf-β1 positive conical keratocytes were observed. Infiltrated inflammation cells were aggregated in the anterior part and were positive for Tgf-β1 (FIG. 32C). On day 7, well regenerated epithelium and corneal stroma were observed in the rTGF-β (GBP1201)-treated cornea (FIG. 32F), and almost the same cells as normal cells were observed in the cornea. The control group (FIG. 32E) showed marked cell hyperplasia (estimated to be proliferated corneal keratocytes and transdifferentiated myofibroblasts) and a strong immune response against TGF-β1 in the corneal stromal layer.

8. Distribution of Fluorescence-Labeled Human hTGF-β (GBP1105) PIP in In Vitro Retina Cells

### I. Material and Method

(A) 3×10⁴APRE-19 cells (human retinal pigment epithelium-derived cells) were seeded in each well of a 6-well plate and cultured in a D-MEM/F-12 medium containing 2 ml of 10% FBS (Invitrogen) at 37° C. in 5% CO₂. After 24 hours of culture, fluorescence-labeled FITC-bound human hTGF-β (GBP1105) was added to the APRE-19 cells at a final concentration of 5 μM in a growth medium, and the mixture was cultured for 30 minutes or 2 or 6 hours. The cells were washed, and an FBS-free medium was added. Viable cells were observed with ×20 magnification and immobilized with 4% paraformaldehyde for 10 minutes. The nucleus was stained with Hoechst3342 (Invitrogen Life Technologies, Corp., Carlsbad, Calif.) and observed again.

### II. Results

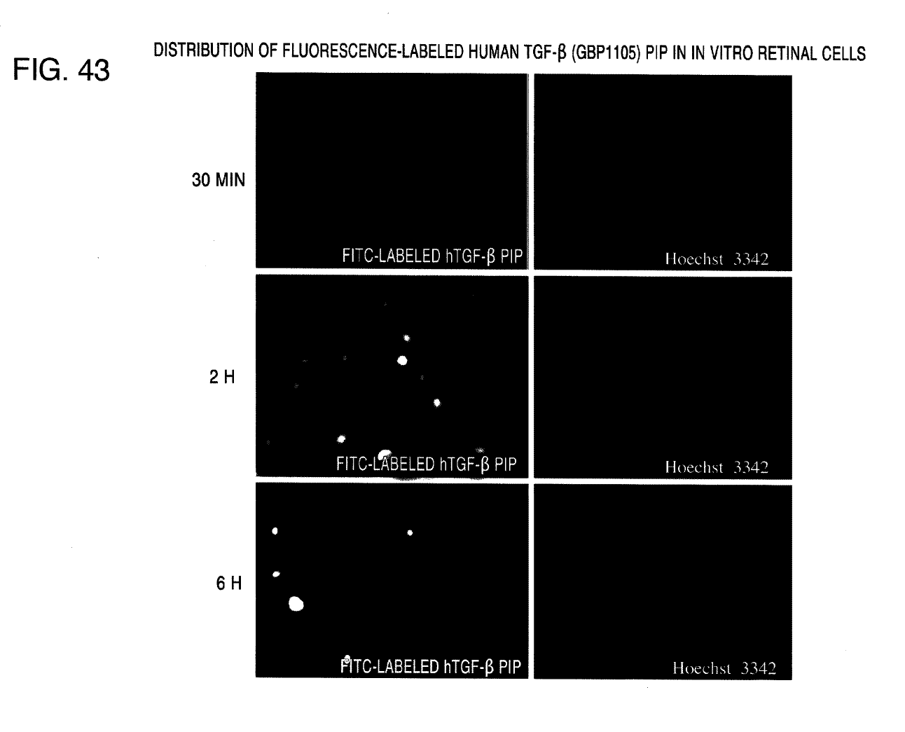

Localization of the FITC-bound human hTGF-β (GBP1105) PIP was not clear at 30 minutes after added to the growth medium in which human retinal pigment epithelium cells APRE-19 cells were cultured. However, localization in the nucleus of all cells was observed in 2 hours. It was demonstrated that the FITC-bound human hTGF-β (GBP1105) PIP remained in the nucleus of APRE-19 cells, the human retinal pigment epithelium cells even at 6 hours after the addition (FIG. 43). From the above findings, it is expected that GBP1105 known to have the expression inhibitory action of human hTGF-β is taken up by human ocular tissues (retinal pigment epithelium cells).

### 9. Statistical Analysis

The results were expressed with mean±SE. Statistical significance was evaluated using Student's t-test and determined when the p value was lower than 0.05.

### INDUSTRIAL APPLICABILITY

The present invention relates to a topical therapeutic agent for an ophthalmic disease containing a compound capable of binding specifically to a DNA sequence. More specifically, the present invention relates to a topical therapeutic agent for an ophthalmic disease containing a pyrrole-imidazole polyamide (hereinafter, also referred to as PIP) has a specific structure.

Sequence listing free text

| | |
|---|---|
| SEQ ID NO: 19 | Forward primer |
| SEQ ID NO: 20 | Reverse primer |
| SEQ ID NO: 21 | Forward primer |
| SEQ ID NO: 22 | Reverse primer |
| SEQ ID NO: 23 | Forward primer |
| SEQ ID NO: 24 | Reverse primer |

1. A topical therapeutic agent for an ophthalmic disease containing a pyrrole-imidazole polyamide comprising an N-methylpyrrole unit (hereinafter, also referred to as Py), an N-methylimidazole unit (hereinafter, also referred to as Im), and a γ-aminobutyric acid unit, wherein the pyrrole-imidazole polyamide can be folded at the γ-aminobutyric acid unit to form a U-shaped conformation in a minor groove of a double helical region (hereinafter, also referred to as a target region) which comprises a part or a whole of a nucleotide sequence from −555 to −528 (SEQ ID NO: 2), a nucleotide sequence from −427 to −399 (SEQ ID NO: 4), or a nucleotide sequence from −384 to −355 (SEQ ID NO: 6) in a human transforming growth factor β (hereinafter, also referred to as TGF-β) gene promoter, and a strand complementary thereto, wherein a Py/Im pair corresponds to a C-G base pair, an Im/Py pair corresponds to a G-C base pair, and a Py/Py pair corresponds to both an A-T base pair and a T-A base pair.

2. The topical therapeutic agent for an ophthalmic disease according to claim 1, further comprising a β-alanine unit.

3. The topical therapeutic agent for an ophthalmic disease according to claim 1 or 2, further comprising a fluorescein isothiocyanate unit.

4. The topical therapeutic agent for an ophthalmic disease according to claim 1, wherein the ophthalmic disease is a trauma due to an alkali burn or a trauma after a corneal surgery.

5. The topical therapeutic agent for an ophthalmic disease according to claim 1, wherein the ophthalmic disease is a proliferative ophthalmic disease or an inflammatory ophthalmic disease.

6. The topical therapeutic agent for an ophthalmic disease according to claim 1, which is in the form of an eye drop.

7. The topical therapeutic agent for an ophthalmic disease according to claim 1, wherein the target region is a double helical region which comprises a part or a whole of a nucleotide sequence from −544 to −538 (SEQ ID NO: 3), a nucleotide sequence from −416 to −410 (SEQ ID NO: 5), or a nucleotide sequence from −373 to −366 (SEQ ID NO: 7) in the transforming growth factor β promoter, and a strand complementary thereto.

8. The topical therapeutic agent for an ophthalmic disease according to claim 1, wherein the pyrrole-imidazole polyamide is represented by the following formula.

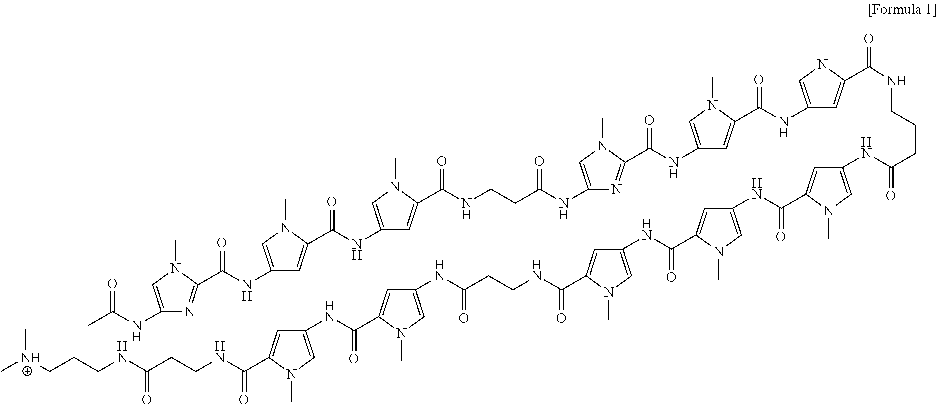

9. The topical therapeutic agent for an ophthalmic disease according to claim 1, wherein the pyrrole-imidazole polyamide is represented by the following formula.

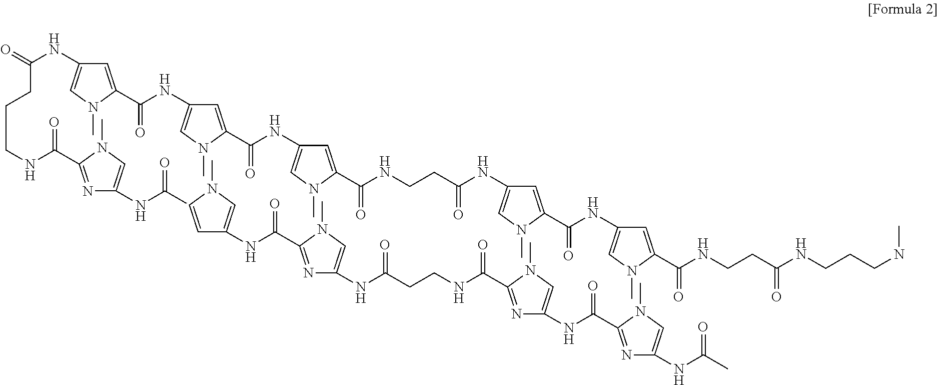

10. The topical therapeutic agent for an ophthalmic disease according to claim 1, wherein the pyrrole-imidazole polyamide is represented by the following formula.

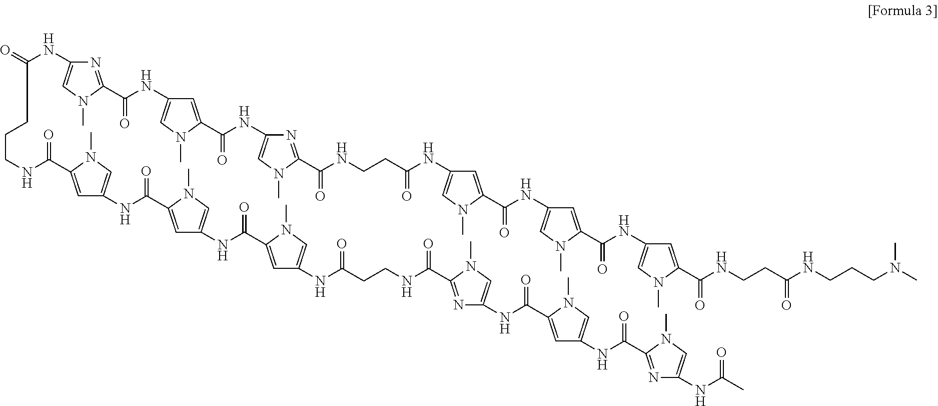

11. A topical therapeutic agent for an ophthalmic disease containing a pyrrole-imidazole polyamide represented by the following formula.

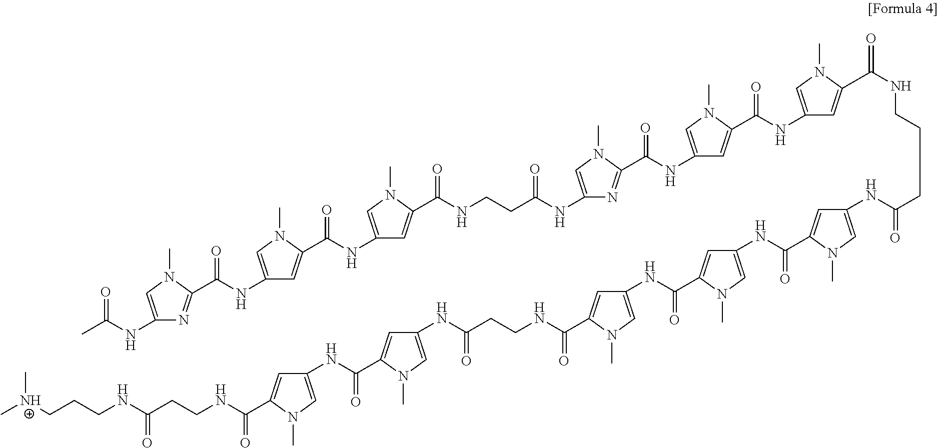

12. A topical therapeutic agent for an ophthalmic disease containing a pyrrole-imidazole polyamide represented by the following formula.

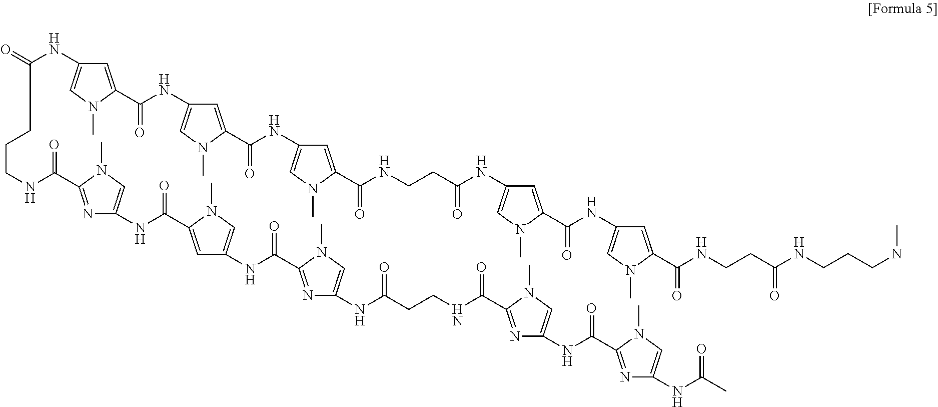

13. A topical therapeutic agent for an ophthalmic disease containing a pyrrole-imidazole polyamide represented by the following formula.

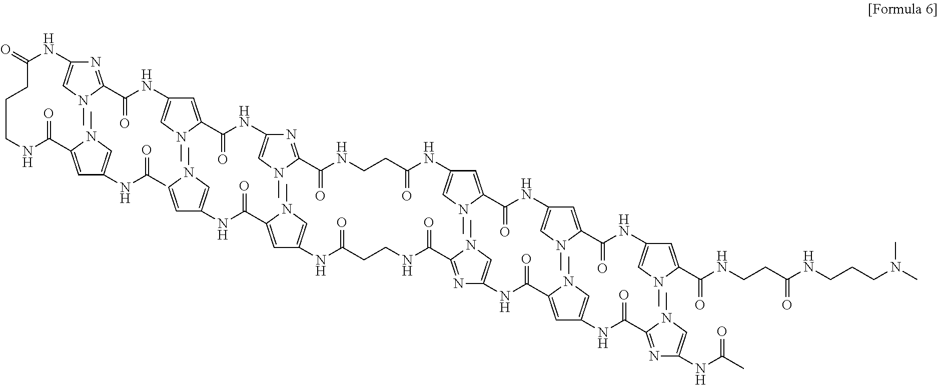

14. A topical therapeutic agent for an ophthalmic disease containing a pyrrole-imidazole polyamide comprising an N-methylpyrrole unit (hereinafter, also referred to as Py), an N-methylimidazole unit (hereinafter, also referred to as Im), and a γ-aminobutyric acid unit, wherein the pyrrole-imidazole polyamide can be folded at the γ-aminobutyric acid unit to form a U-shaped conformation in a minor groove of a double helical region (hereinafter, also referred to as a target region) which comprises a part or a whole of a nucleotide sequence from −2316 to −2287 (SEQ ID NO: 25) or a nucleotide sequence from −2322 to −2293 (SEQ ID NO: 8) in a rat transforming growth factor β (hereinafter, also referred to as TGF-β) gene promoter, and a strand complementary thereto, wherein a Py/Im pair corresponds to a C-G base pair, an Im/Py pair corresponds to a G-C base pair, and a Py/Py pair corresponds to both an A-T base pair and a T-A base pair.

15. The topical therapeutic agent for an ophthalmic disease according to claim 14, further comprising a β-alanine unit.

16. The topical therapeutic agent for an ophthalmic disease according to claim 14 or 15, further comprising a fluorescein isothiocyanate unit.

17. The topical therapeutic agent for an ophthalmic disease according to claim 14, wherein the ophthalmic disease is a trauma due to an alkali burn or a trauma after a corneal surgery.

18. The topical therapeutic agent for an ophthalmic disease according to claim 14, wherein the ophthalmic disease is a proliferative ophthalmic disease or an inflammatory ophthalmic disease.

19. The topical therapeutic agent for an ophthalmic disease according to claim 14, which is in the form of an eye drop.

20. The topical therapeutic agent for an ophthalmic disease according to claim 14, wherein the target region is a double helical region which comprises a part or a whole of a nucleotide sequence from −2305 to −2298 (SEQ ID NO: 26) or a nucleotide sequence from −2311 to −2304 (SEQ ID NO: 9) in the transforming growth factor β promoter, and a strand complementary thereto.

21. The topical therapeutic agent for an ophthalmic disease according to claim 14, wherein the pyrrole-imidazole polyamide is represented by the following formula.

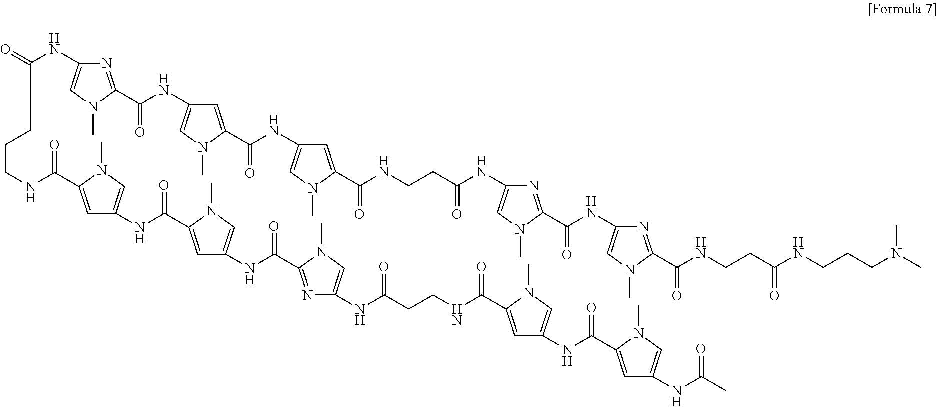

22. The topical therapeutic agent for an ophthalmic disease according to claim 14, wherein the pyrrole-imidazole polyamide is represented by the following formula.

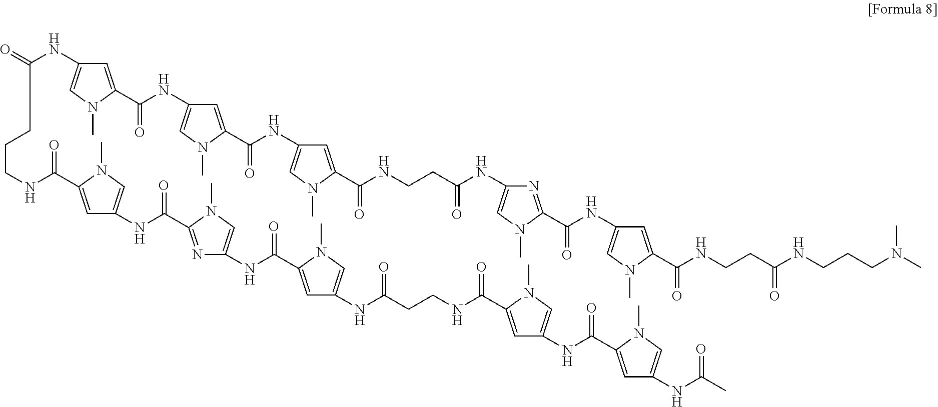

23. A topical therapeutic agent for an ophthalmic disease containing a pyrrole-imidazole polyamide represented by the following formula.

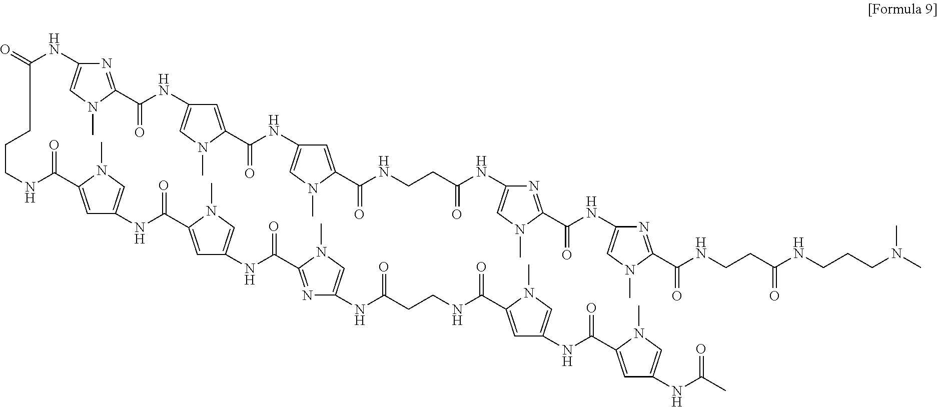

24. A topical therapeutic agent for an ophthalmic disease containing a pyrrole-imidazole polyamide represented by the following formula.

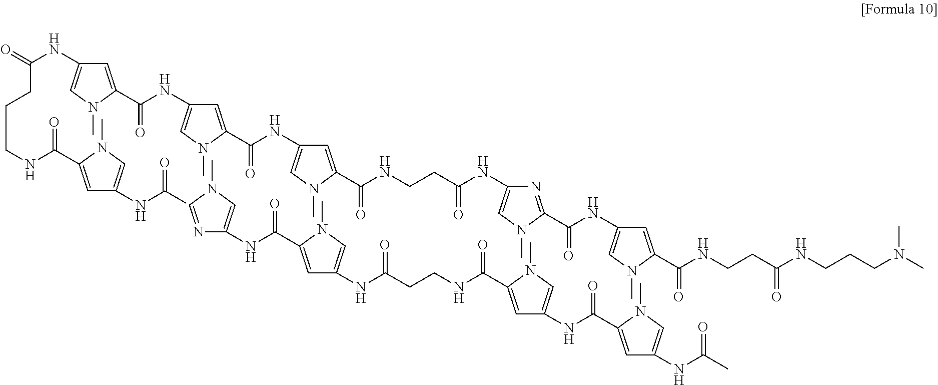

25. A topical therapeutic agent for an ophthalmic disease containing a pyrrole-imidazole polyamide comprising an N-methylpyrrole unit (hereinafter, also referred to as Py), an N-methylimidazole unit (hereinafter, also referred to as Im), and a γ-aminobutyric acid unit, wherein the pyrrole-imidazole polyamide can be folded at the γ-aminobutyric acid unit to form a U-shaped conformation in a minor groove of a double helical region (hereinafter, also referred to as a target region) which comprises a part or a whole of a nucleotide sequence from −88 to −59 (SEQ ID NO: 11) or a nucleotide sequence from −616 to −588 (SEQ ID NO: 13) in a human matrix metalloprotease 9 (hereinafter, also referred to as hMMP-9) gene promoter, and a strand complementary thereto, wherein a Py/Im pair corresponds to a C-G base pair, an Im/Py pair corresponds to a G-C base pair, and a Py/Py pair corresponds to both an A-T base pair and a T-A base pair.

26. The topical therapeutic agent for an ophthalmic disease according to claim 25, further comprising a β-alanine unit.

27. The topical therapeutic agent for an ophthalmic disease according to claim 25 or 26, further comprising a fluorescein isothiocyanate unit.

28. The topical therapeutic agent for an ophthalmic disease according to claim 25, wherein the ophthalmic disease is a trauma due to an alkali burn or a trauma after a corneal surgery.

29. The topical therapeutic agent for an ophthalmic disease according to claim 25, wherein the ophthalmic disease is a proliferative ophthalmic disease or an inflammatory ophthalmic disease.

30. The topical therapeutic agent for an ophthalmic disease according to claim 25, which is in the form of an eye drop.

31. The topical therapeutic agent for an ophthalmic disease according to claim 25, wherein the target region is a double helical region which comprises a part or a whole of a nucleotide sequence from −77 to −70 (SEQ ID NO: 12) or a nucleotide sequence from −605 to −599 (SEQ ID NO: 14) in a transforming growth factor β promoter, and a strand complementary thereto.

32. The topical therapeutic agent for an ophthalmic disease according to claim 25, wherein the pyrrole-imidazole polyamide is represented by the following formula.

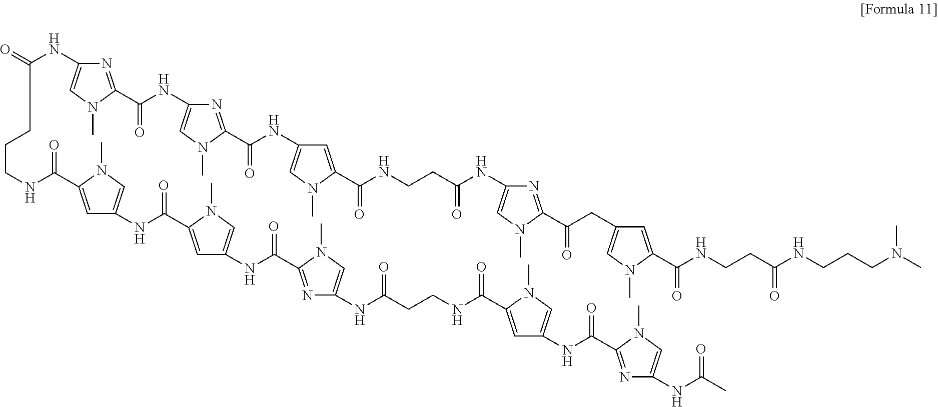

33. The topical therapeutic agent for an ophthalmic disease according to claim 25, wherein the pyrrole-imidazole polyamide is represented by the following formula.

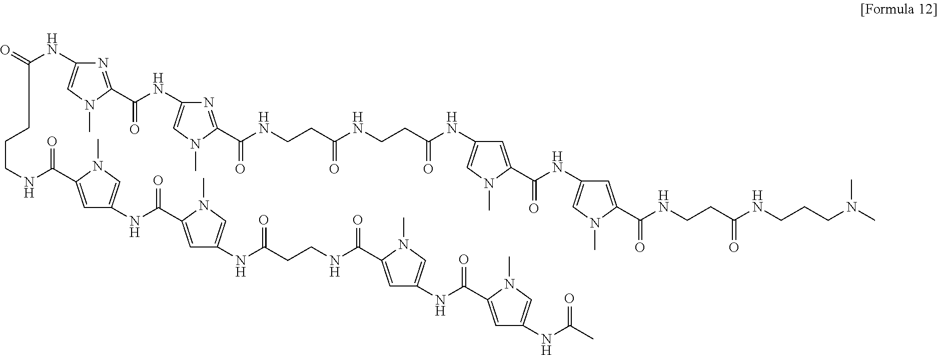

34. A topical therapeutic agent for an ophthalmic disease containing a pyrrole-imidazole polyamide represented by the following formula.

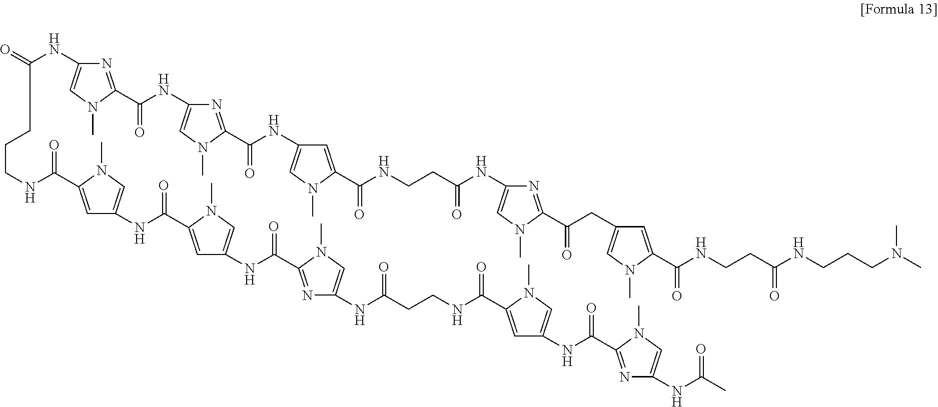

35. A topical therapeutic agent for an ophthalmic disease containing a pyrrole-imidazole polyamide represented by the following formula.

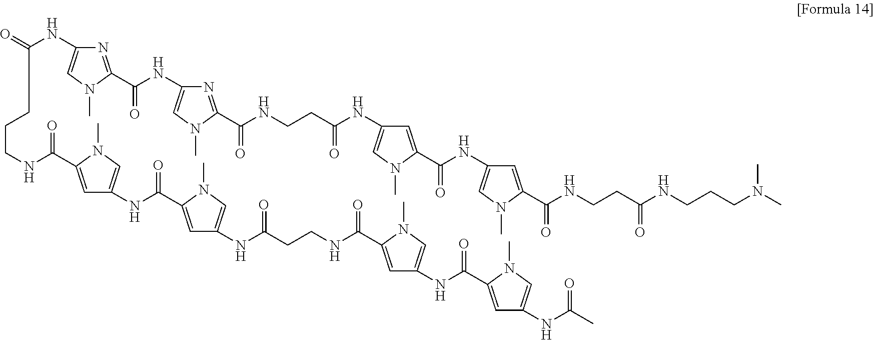

36. A topical therapeutic agent for an ophthalmic disease containing a pyrrole-imidazole polyamide comprising an N-methylpyrrole unit (hereinafter, also referred to as Py), an N-methylimidazole unit (hereinafter, also referred to as Im), and a γ-aminobutyric acid unit, wherein the pyrrole-imidazole polyamide can be folded at the γ-aminobutyric acid unit to form a U-shaped conformation in a minor groove of a double helical region (hereinafter, also referred to as a target region) which comprises a part or a whole of a nucleotide sequence from −105 to −76 (SEQ ID NO: 15) or a nucleotide sequence from −602 to −575 (SEQ ID NO: 17) in a rat matrix metalloprotease 9 (hereinafter, also referred to as rMMP-9) gene promoter, and a strand complementary thereto, wherein a Py/Im pair corresponds to a C-G base pair, an Im/Py pair corresponds to a G-C base pair, and a Py/Py pair corresponds to both an A-T base pair and a T-A base pair.

37. The topical therapeutic agent for an ophthalmic disease according to claim 36, further comprising a β-alanine unit.

38. The topical therapeutic agent for an ophthalmic disease according to claim 36 or 37, further comprising a fluorescein isothiocyanate unit.

39. The topical therapeutic agent for an ophthalmic disease according to claim 36, wherein the ophthalmic disease is a trauma due to an alkali burn or a trauma after a corneal surgery.

40. The topical therapeutic agent for an ophthalmic disease according to claim 36, wherein the ophthalmic disease is a proliferative ophthalmic disease or an inflammatory ophthalmic disease.

41. The topical therapeutic agent for an ophthalmic disease according to claim 36, which is in the form of an eye drop.

42. The topical therapeutic agent for an ophthalmic disease according to claim 36, wherein the target region is a double helical region which comprises a part or a whole of a nucleotide sequence from −94 to −87 (SEQ ID NO: 16) or a nucleotide sequence from −591 to −586 (SEQ ID NO: 18) in the matrix metalloprotease 9 promoter, and a strand complementary thereto.

43. The topical therapeutic agent for an ophthalmic disease according to claim 36, wherein the pyrrole-imidazole polyamide is represented by the following formula.

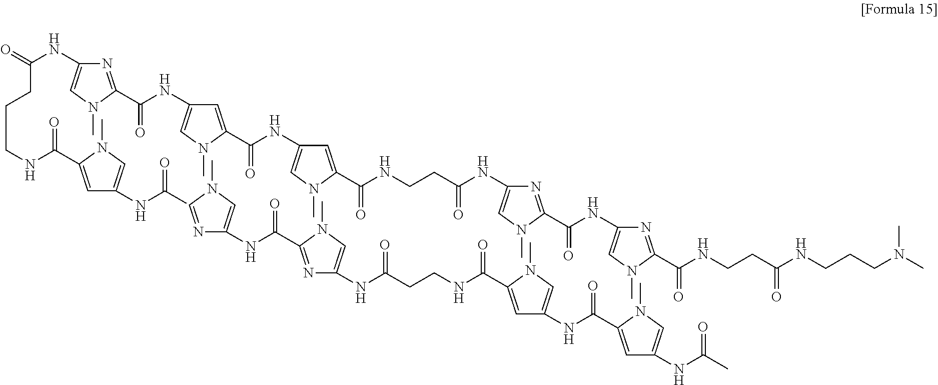

44. The topical therapeutic agent for an ophthalmic disease according to claim 36, wherein the pyrrole-imidazole polyamide is represented by the following formula.

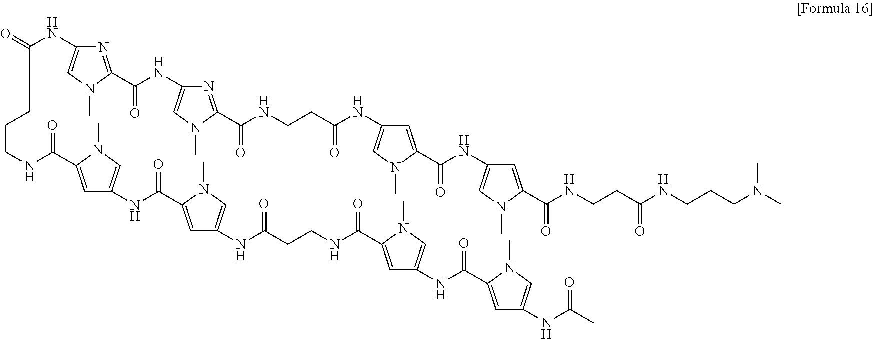

45. A topical therapeutic agent for an ophthalmic disease containing a pyrrole-imidazole polyamide represented by the following formula.

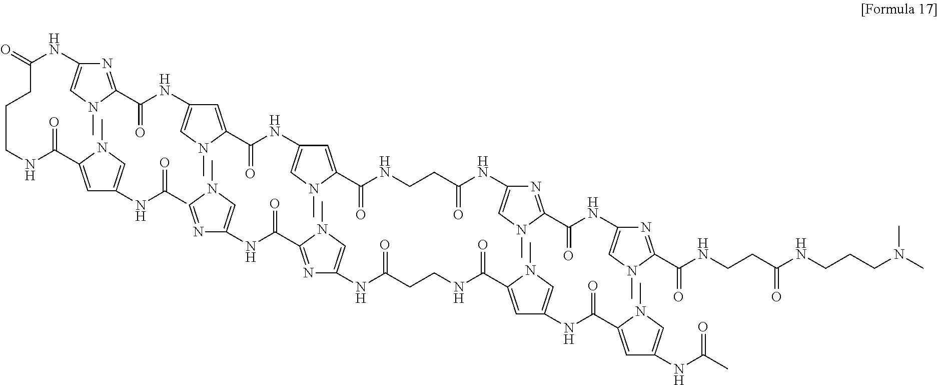

46. A topical therapeutic agent for an ophthalmic disease containing a pyrrole-imidazole polyamide represented by the following formula. [chemical structure]
